SEQUENCE LISTING (5 sequences)

SEQ ID NO: 1 - protein, 77 aa, Homo sapiens
AVLPRSAKELRCQCIKTYSKPFHPKFIKELRVIESGPHCANTEIIVKLSDGRELCLDPKENWVQRVVEKFLKRAENS

SEQ ID NO: 2 - protein, 72 aa, Homo sapiens
SAKELRCQCIKTYSKPFHPKFIKELRVIESGPHCANTEIIVKLSDGRELCLDPKENWVQRVVEKFLKRAENS

SEQ ID NO: 3 - DNA, 21 bp, Artificial Sequence
  Description of Artificial Sequence Synthetic oligonucleotide
aannannnnnnunnhnnnnnw

SEQ ID NO: 4 - DNA, 21 bp, Artificial Sequence
  Description of Artificial Sequence Synthetic oligonucleotide
wnnnnndnnannnnnnunntt

SEQ ID NO: 5 - DNA, 21 bp, Artificial Sequence
  Description of Artificial Sequence Synthetic oligonucleotide
nnannnnnnunnhnnnnnwtt

(19) United States
(12) Patent Application Publication
(10) Pub. No.: US 2014/0212380 A1
(43) Pub. Date: Jul. 31, 2014

(54) TARGETS AND AGENTS FOR THE TREATMENT OF IMPAIRED BONE FRACTURE HEALING

(71) Applicants: Valerie Gangji, Rhode-Saint-Genese (BE); Jean-Philippe Hauzeur, Bruxelles (BE); Dominique De Seny, Liege (BE); Myrielle Mathieu, Dilbeek (BE); Aude Ingels, Louvain-La-Neuve (BE); Sabrina Rigutto, Tollembeek (BE); Delphine Spruyt, Gilly (BE); Enrico Bastianelli, Rhode-Saint-Genese (BE); Valentina Albarani, Bruxelles (BE); Xavier Pesesse, Bierghes (BE); Michel Malaise, Grivegnee (BE)

(72) Inventors: Valerie Gangji, Rhode-Saint-Genese (BE); Jean-Philippe Hauzeur, Bruxelles (BE); Dominique De Seny, Liege (BE); Myrielle Mathieu, Dilbeek (BE); Aude Ingels, Louvain-La-Neuve (BE); Sabrina Rigutto, Tollembeek (BE); Delphine Spruyt, Gilly (BE); Enrico Bastianelli, Rhode-Saint-Genese (BE); Valentina Albarani, Bruxelles (BE); Xavier Pesesse, Bierghes (BE); Michel Malaise, Grivegnee (BE)

(73) Assignees: Universite Libre De Bruxelles, Bruxelles (BE); Centre Hospitalier Universitaire de Liege, Liege (BE); Universite de Liege, Liege (BE); Bone Therapeutics SA, Gosselies (BE)

(21) Appl. No.: 14/124,165

(22) PCT Filed: Jun. 11, 2012
(86) PCT No.: PCT/EP2012/061036

§ 371 Date: Dec. 5, 2013

(30) Foreign Application Priority Data
Jun. 10, 2011 (EP) 11169605.0

(51) Int. Cl. C07K 14/54

(52) U.S. Cl. CPC C07K 14/5421
USPC 424/85.2

(57) ABSTRACT

The application teaches a composition comprising one or more pharmaceutical active ingredients selected from the group consisting of interleukin-8 (IL-8 or CXCL8), a functional fragment of IL-8, a functional variant of IL-8, and an agonist of IL-8 receptor, for therapeutic and/orprophylactic interventions in impaired bone fracture healing, such as but not limited to non-union fractures, mal-union fractures, or delayed union fractures.

### FIELD OF THE INVENTION

The invention is in the medical field, more precisely in the field of new therapeutic targets, agents and methods, more particularly targets, agents and methods useful in the treatment of impaired bone fracture healing, such as but not limited to non-union fractures, mal-union fractures or delayed union fractures. The invention also concerns methods for identifying agents modulating the level and/or activity of targets useful in the treatment of impaired bone fracture healing.

### BACKGROUND OF THE INVENTION

Impaired fracture healing encompasses any anomalies and deficiencies of bone fracture healing such as inadequate, delayed or absent bone fracture healing, including without limitation mal-unions, delayed unions and non-unions. Non-union fractures, also known as non-unions (NU), including inter alia tight non-unions and unstable non-unions (pseudarthrosis), are characterised by a failure of fracture repair processes, without hope of spontaneous healing. The reported rate of non-unions varies between 2% and 10% of all fractures, depending on the authors (Gaston et al. J. Bone Joint Surg. Br., 2007, vol. 89(12), 1553-1560; Tzioupis and Giannoudis. Injury, 2007, vol. 38 Suppl 2, S3-S9). Non-unions may be classified as hypertrophic or oligotrophic if bony fragment sites are vascular. Hypertrophic non-unions are usually explained by an instability at the fracture site. The oligotrophic non-unions typically occur after major displacement of the fracture sites and present an inadequate healing response as shown by the absence of callus. In non-unions classified as atrophic, the bony fragments are avascular, adynamic and incapable of biologic reaction (Frolke et al. Injury, 2007, vol. 38 Suppl 2, S19-S22).

Mal-unions are characterized by an imperfect union of previously fragmented bone. A delayed union can be defined as a fracture in which healing has not occurred in the expected time and the outcome remains uncertain.

In normal healing process, a bone fracture initiates a sequence of inflammation, repair, and remodelling that can restore the injured bone to its original state. In humans, the inflammatory phase lasts about 5 to 7 days and begins with the development of a haematoma and is followed by the invasion of inflammatory cells. These cells, in association with the local cells, secrete cytokines, chemokines and growth factors to promote the recruitment of osteogenic progenitor cells and endothelial progenitor cells, essential to initiate the repair process (Einhorn. Clin. Orthop. Relat. Res., 1998, vol. 355 Suppl: S7-21). The recruitment of progenitor cells is divided in four phases: mobilisation, migration, invasion and engraftment of the cells to the fracture site. Impairment of inter alia any one or more of the above processes can result in impaired bone fracture healing.

Treatment of impaired bone fracture healing typically relies on orthopaedic surgical interventions comprising or chosen from for example the removal of infection, the removal of scar tissue from between the fracture fragments, immobilisation of the fracture using bone fixation devices (such as metal plates, rods, pins, nails, wires, etc.), external fixators or Ilizarov device, introduction of gap refill materials, and/or interposition of bone grafts (such as cancellous- or corticocancellous-bone grafting). These surgical interventions may be associated with severe adverse events leading to long-time and expensive hospitalizations. Moreover, while a drastic measure, amputation may be warranted if a functional limb cannot be achieved. Therefore, there is a need for novel, non-invasive and safe therapies to stimulate bone fracture healing.

Pharmaceutical or biological approaches to the treatment of impaired fracture healing are at best sparse. Local biologic stimulation therapies include local stem cells (mainly bone marrow-derived stem cells) injections, growth factors injections or platelet rich plasma injection. In situ injection of growth factors is promising. For example, Dimitriou et al. Injury, 2005, vol. 36, suppl. 4, S51-9, evaluated the efficacy and safety of recombinant bone morphogenetic protein 7 (BMP-7) as a bone-stimulating agent in the treatment of persistent fracture non-unions. Calori et al. Injury, 2008, vol. 39, 1391-402, concluded that in the treatment of persistent long bone non-unions, the application of recombinant BMP-7 as a bone-stimulating agent is superior compared to that of platelet-rich plasma with regard to their clinical and radiological efficacy. BMP-2 has also been proved to enhance bone repair in clinical trials (Govender et al. J. Bone Joint Surg. Am., 2002, vol. 84, 2123-34). Yet, if these molecules are already used in clinic, they might be associated with adverse events, such as ectopic bone formation or excessive soft tissue swelling, and are expensive therapeutic agents. Several preclinical reports have shown beneficial effects of PDGF treatment on bone fracture healing. For example, Hollinger et al. have shown an enhanced bone healing in patients who received local injection of PDGF (J. Bone Joint Surg. Am., 2008, vol. 90, Suppl 1:48-54). Moreover, prospective, randomized, controlled clinical trials have shown that Augment™ Bone Graft (rhPDGF-BB/β-TCP) was comparable to autograft in foot and ankle fusion surgery. Yet, other studies have failed to show the clinical usefulness of isolated percutaneous platelet gel supplementation in long bone non-unions (Mariconda et al. J. Orthop Trauma, 2008, vol. 22(5), 342-5). Therefore, new molecules need to be investigated to enhance bone fracture healing, enhance patient care and reduce the cost of long-lasting hospitalisations (Marsell et al. J. Orthop. Trauma, 2010, vol. 24, S4-S8).

Consequently, there exists a continuous need for additional and preferably improved therapeutic targets, agents and methods useful in the treatment of non-union fractures. Targets may include for example biological molecules, such as proteins or polypeptides.

### SUMMARY OF THE INVENTION

Having conducted extensive experiments and tests, the inventors identified biological molecules whose levels are significantly altered in impaired healing of bone fractures compared to healthy subjects, and which thus constitute useful and promising targets for prophylactic and/or therapeutic interventions in impaired fracture healing. The synonymous phrases “impaired bone fracture healing” or “impaired fracture healing” as used herein encompass any anomalies, abnormalities and deficiencies of bone fracture healing, such as inadequate, delayed or absent bone fracture healing. The phrases intend to specifically comprise and preferably denote mal-unions, delayed unions and non-unions, more preferably denote non-unions, including inter alia tight non-unions and unstable non-unions (pseudarthrosis).

Expanding on these findings, the inventors recognised stromal derived factor-1 (SDF-1 or CXCL12), SDF-1 receptor, interleukin-8 (IL-8 or CXCL8), IL-8 receptor, interleukin-6 (IL-6) or IL-6 receptor as valuable targets for therapeutic and/or prophylactic interventions in impaired fracture healing. The inventors thus also contemplate these molecules as useful targets for therapeutic and/or prophylactic interventions in the treatment of any fractures.

The inventors realised that modulating the level and/or activity of SDF-1, SDF-1 receptor (preferably any one or both of CXCR4 and CXCR7), IL-8, IL-8 receptor (preferably any one or both of CXCR1 and CXCR2), IL-6 and/or IL-6 receptor (CD126) in subjects suffering from impaired fracture healing or in subjects having any fracture constituted a valuable option for treating such subjects. The inventors further recognised the importance of screening for and identifying agents capable of modulating the level and/or activity of SDF-1, SDF-1 receptor, IL-8, IL-8 receptor, IL-6 and/or IL-6 receptor in order to provide or select those agents useful in treating impaired fracture healing or in treating a fracture.

Following extensive research, the inventors recognised compositions comprising one or more pharmaceutical active ingredients selected from the group consisting of IL-8, a functional fragment of IL-8, a functional variant of IL-8, and an agonist of IL-8 receptor, as a useful option for treating impaired bone fracture healing.

Thus, among others the following aspects and embodiments are provided in accordance with the present invention:

An agent that is able to modulate, such as increase or reduce, the level and/or activity of any one or more nucleic acids or proteins selected from the group consisting of SDF-1, SDF-1 receptor, IL-8, IL-8 receptor, IL-6 and IL-6 receptor, for use as a medicament.

An agent that is able to modulate, such as increase or reduce, the level and/or activity of any one or more nucleic acids or proteins selected from the group consisting of SDF-1, SDF-1 receptor, IL-8, IL-8 receptor, IL-6 and IL-6 receptor, for use in the treatment of impaired fracture healing or for use in the treatment of a fracture. The phrase “for use in the treatment of” is intended as synonymous with phrases “for use in treating” and “for use in a method for treatment of”.

Use of an agent that is able to modulate, such as increase or reduce, the level and/or activity of any one or more nucleic acids or proteins selected from the group consisting of SDF-1, SDF-1 receptor, IL-8, IL-8 receptor, IL-6 and IL-6 receptor for the manufacture of a medicament for the treatment of impaired fracture healing or for the treatment of a fracture.

Use of an agent that is able to modulate, such as increase or reduce, the level and/or activity of any one or more nucleic acids or proteins selected from the group consisting of SDF-1, SDF-1 receptor, IL-8, IL-8 receptor, IL-6 and IL-6 receptor for the treatment of impaired fracture healing or for the treatment of a fracture.

A method for treating impaired fracture healing in a subject in need of such treatment or for treating a fracture in a subject in need of such treatment, comprising administering to said subject a therapeutically or prophylactically effective amount of an agent that is able to modulate, such as increase or reduce, the level and/or activity of any one or more nucleic acids or proteins selected from the group consisting of SDF-1, SDF-1 receptor, IL-8, IL-8 receptor, IL-6 and IL-6 receptor.

An assay to select or isolate, from a group of test agents, a candidate agent potentially useful in the treatment of impaired fracture healing or in the treatment of a fracture, said assay comprising determining whether a test agent can modulate, such as increase or reduce, the level and/or activity of any one or more nucleic acids or proteins selected from the group consisting of SDF-1, SDF-1 receptor, IL-8, IL-8 receptor, IL-6 and IL-6 receptor.

An assay to select or isolate, from a group of test agents, a candidate agent potentially useful in the treatment of impaired fracture healing or in the treatment of a fracture, said assay comprising determining whether a test agent can specifically bind to any one or more nucleic acids or proteins selected from the group consisting of SDF-1, SDF-1 receptor, IL-8, IL-8 receptor, IL-6 and IL-6 receptor. Such binding agents may be particularly suited or promising for modulating the respective nucleic acids or proteins.

Any one of the aforementioned assays further comprising use of the selected or isolated agent for the preparation of a composition for administration to and monitoring the prophylactic and/or therapeutic effect thereof in a non-human animal model, preferably a non-human mammal model of impaired fracture healing.

An agent selected or isolated by the aforementioned assay.

A pharmaceutical composition or formulation comprising a prophylactically and/or therapeutically effective amount of one or more agents selected or isolated by the aforementioned assay, or a pharmaceutically acceptable N-oxide form, addition salt, prodrug or solvate thereof, and further comprising one or more of pharmaceutically acceptable carriers.

A method for producing aforementioned pharmaceutical composition or formulation as, comprising admixing said one or more agents with said one or more pharmaceutically acceptable carriers.

Preferably, an aspect of the present invention relates to a composition comprising, consisting essentially of, or consisting of one or more pharmaceutical active ingredients selected from the group consisting of IL-8, a functional fragment of IL-8, a functional variant of IL-8, and an agonist of IL-8 receptor, for use in the treatment of impaired bone fracture healing.

Compositions as defined herein for use in the treatment of impaired bone fracture healing are advantageous inter alia because these compositions allow efficient treatment of the impaired bone fracture healing such as a non-union fracture. Furthermore, such compositions advantageously allow percutaneous administration thereby overcoming the need for invasive surgical interventions. Hence, the present compositions advantageously provide increased patient compliance in the treatment of impaired bone fracture healing.

As mentioned above, in normal bone healing processes, a bone fracture initiates a sequence of inflammation, repair, and remodelling that can restore the injured bone to its original state. However, in patients with impaired bone fracture healing, these fracture repair processes are absent and patients will not heal spontaneously. Due to the differences between bone fractures the healing of which proceeds normally and impaired bone fracture healing such as non-union fractures, it is unexpected that the compositions as defined herein allow treatment of impaired bone fracture healing such as a non-union fracture.

Also provided in certain embodiments is the use of a composition comprising, consisting essentially of, or consisting of one or more pharmaceutical active ingredients selected from the group consisting of IL-8, a functional fragment of IL-8, a functional variant of IL-8, and an agonist of IL-8 receptor, for the manufacture of a medicament for the treatment of impaired bone fracture healing. Such treatment may typically involve percutaneous administration of the composition.

Further provided in certain embodiments is the use of a composition comprising, consisting essentially of, or consisting of one or more pharmaceutical active ingredients selected from the group consisting of IL-8, a functional fragment of IL-8, a functional variant of IL-8, and an agonist of IL-8 receptor, for the treatment of impaired bone fracture healing.

Also intended in certain embodiments is a method for treating impaired bone fracture healing in a subject in need of such treatment, comprising administering to said subject a therapeutically or prophylactically effective amount of a composition comprising, consisting essentially of, or consisting of one or more pharmaceutical active ingredients selected from the group consisting of IL-8, a functional fragment of IL-8, a functional variant of IL-8, and an agonist of IL-8 receptor.

In preferred embodiments, the impaired bone fracture healing may be selected from the group consisting of non-union fracture, mal-union fracture, and delayed union fracture.

The one or more pharmaceutically active ingredients may be isolated or recombinant (preferably native human) IL-8 (CXCL-8), isolated or recombinant (preferably native human) IL-8 peptide, or a functional variant thereof. For example, the one or more pharmaceutically active ingredients may be human recombinant IL-8 peptide, such as a 77-amino acid IL-8 peptide having the amino acid sequence of SEQ ID No. 1, a 72-amino acid IL-8 peptide having the amino acid sequence of SEQ ID No. 2, or a 78-amino acid IL-8 peptide purified from Escherichia coli as described by Lindley et al. PNAS, 1988, vol. 85(23), 9199-9203.

In certain embodiments, the one or more pharmaceutical active ingredients may be an IL-8 peptide or a functional variant thereof, wherein the IL-8 peptide comprises an amino acid sequence selected from SEQ ID No. 1 or SEQ ID No. 2.

In certain embodiments, the present compositions may further comprise a gel-forming material. The terms “gel-forming”, “one phase” or “monophasic” can be used interchangeably herein. The term “gel-forming material” as intended throughout this specification encompasses materials forming or capable of forming a solid, jelly-like structure (gel). The gel-forming material may be a gel per se or the gel-forming material may be a material that is not a gel (e.g., that is liquid or solid) and that forms a gel when combined with or exposed to materials and/or conditions conducive to gel formation, for example but without limitation, when dissolved or dispersed in a suitable liquid phase, such as in an aqueous solution or dispersion for instance upon contact of said gel-forming material with physiological or bodily fluids. Hence, a gel-forming material may encompass a material capable of gelifying a liquid phase, such as an aqueous liquid phase.

The gel-forming material may be for example collagen, a glyceride, a glycosaminoglycan, a polysaccharide, gelatine, poly-lactic acid, or poly-lactic glycolic acid.

The term “glyceride”, as used herein, refers to an ester formed from glycerol and one or more of the same or distinct fatty acid(s). The terms “glyceride” and “acylglycerol” can be used interchangeably. The term glyceride encompasses monoglycerides (monoacylglycerol), diglycerides and triglycerides depending on whether one, two, or three fatty acids are esterified with glycerol. The gel-forming material as intended herein can further be a glycerate such as for instance but without limitation oleyl glycerate or phytanyl glycerate.

For example, the glyceride is a monoglyceride. A monoglyceride can be a 1-monoacylglycerol or a 2-monoacylglycerol depending on the position of the ester bond on the glycerol moiety. Non-limiting examples of monoglycerides are for instance glycerol mono(o)leate (GMO), glycerol monolinoleate, glycerol monolinolenate, glycerol monopalmitate, glycerol monostearate or glycerol monolaurate.

For example, the glyceride may be an ester of glycerol and oleic acid. The term “oleic acid” refers to a monounsaturated omega-9 fatty acid, more particularly (9Z)-Octadec-9-enoic acid also known as cis-9-Octadecenoic acid or 18:1 cis-9. For instance, the glyceride may be a monoglyceride with oleic acid, i.e., glycerol monooleate, also commonly denoted as glycerol monoleate, mono(o)lein, glyceryl monooleate, glyceryl oleate, (Z)-1-oleoyl-sn-glycerol, or 1,2,3-propanetriol 9-octadecenoic acid.

The term “glycosaminoglycan”, as used herein, refers to an unbranched polysaccharide consisting of a repeating disaccharide unit. The glycosaminoglycan may be selected from the group consisting of hyaluronic acid and derivatives thereof, a proteoglycan and derivatives thereof, a chondroitin sulfate, a keratan sulfate, a chitosan and derivatives thereof, and a chitin and derivatives thereof.

The term “hyaluronic acid” or “HA” may be used interchangeably with “hyaluronan” or “hyaluronate”. The term “hyaluronic acid” refers to an anionic, non-sulfated polymer of disaccharides composed of D-glucuronic acid and N-acetyl-D-glucosamine, linked via alternating β-1,4 and β-1,3 glycosidic bonds. Hyaluronic acid derivatives include but are not limited to salts of hyaluronate such as sodium hyaluronate or an ester of hyaluronic acid with an alcohol of the aliphatic, heterocyclic or cycloaliphatic series, or a sulphated form of hyaluronic acid or combination of agents containing hyaluronic acid.

The term “proteoglycan” refers to proteins with one or more covalently attached glycosaminoglycan (GAG) chain(s). The glycosaminoglycan can be a proteoglycan selected from decorin, biglycan, testican, fibromodulin, lumican, versican, perlecan, neurocan or aggrecan.

The term “chondroitin sulfate” refers to a polymer of disaccharides composed of N-acetylgalactosamine and glucuronic acid, each of which can be sulfated in variable positions and quantities. The chondroitic sulfate can be selected from chondroitin-4-sulfate, chondroitin-6-sulfate, chondroitin-2,6-sulfate, chondroitin-4,6-sulfate.

The term “keratan sulfate” may be used interchangeably with “keratosulfate” and refers to a polymer of repeating disaccharides—3 Galβ1-4GlcNAcβ1—which can be sulfated at carbon position 6 (C6) of either or both the Gal or GlcNAc monosaccharides.

The term “chitosan” refers to a linear polymer composed of randomly distributed 13-(1-4)-linked D-glucosamine (deacetylated unit) and N-acetyl-D-glucosamine (acetylated unit).

The term “chitin” refers to a polymer composed of β-(1,4)-linked N-acetylglucosamine.

Certain embodiments relate to a composition comprising, consisting essentially of, or consisting of a gel-forming material and one or more pharmaceutical active ingredients selected from the group consisting of IL-8, a functional fragment of IL-8, a functional variant of IL-8, and an agonist of IL-8 receptor, for use in the treatment of impaired bone fracture healing.

In preferred embodiments, the gel-forming material may be collagen and the pharmaceutical active ingredient may be an IL-8 peptide comprising an amino acid sequence selected from SEQ ID No. 1 or SEQ ID No. 2.

In certain embodiments, the composition may be configured for percutaneous administration. Such compositions advantageously increase the ease of administration of the composition without the need for invasive and costly orthopaedic surgical interventions. The recitation “percutaneous administration”, as used herein, refers to any medical administration procedure where access to inner organs or tissue is done via needle-puncture of the skin, such as by injection, rather than by using surgery where inner organs or tissue are exposed.

Accordingly, particularly preferred embodiments provide the composition as described herein for use in the treatment of impaired bone fracture healing by percutaneous administration (i.e., wherein the composition is (to be) administered percutaneously), even more preferably the composition may be administered by percutaneous injection. To this end, such compositions would be configured for percutaneous administration, more preferably would be configured as injectable composition.

Where suitable, the composition may further contain one or more pharmaceutically acceptable carriers/excipients.

In any one or more of the above aspects and/or embodiments, various preferred but non-limiting characteristics may apply, such as:

The agent may be preferably able to increase the level and/or activity of SDF-1 and/or SDF-1 receptor.

The agent may be preferably able to reduce the level and/or activity of IL-8 and/or IL-8 receptor.

The agent may be preferably able to reduce the level and/or activity of IL-6 and/or IL-6 receptor.

The agent may be preferably able to specifically bind to any one or more nucleic acids or proteins selected from the group consisting of SDF-1, SDF-1 receptor, IL-8, IL-8 receptor, IL-6 and IL-6 receptor.

The agent may preferably comprise, consist essentially of or consist of, i.e., the agent may be preferably selected from a group consisting of, an antibody or a fragment or derivative thereof, a protein or polypeptide, a peptide, a peptidomimetic, an aptamer, a photoaptamer, a nucleic acid, or a chemical substance, preferably an organic molecule, more preferably a small organic molecule.

The agent may be able to increase the expression of said one or more nucleic acids or proteins selected from the group consisting of SDF-1, SDF-1 receptor, IL-8, IL-8 receptor, IL-6 and IL-6 receptor. For example, such agent may comprise, consist essentially of or consist of a recombinant nucleic acid comprising a sequence encoding any one or more of SDF-1, SDF-1 receptor, IL-8, IL-8 receptor, IL-6 and IL-6 receptor operably linked to one or more regulatory sequences allowing for expression of said sequence or sequences encoding any one or more of SDF-1, SDF-1 receptor, IL-8, IL-8 receptor, IL-6 and IL-6 receptor. Introduction (e.g., by transfection or transduction) of such agent to a subject shall effect expression of any one or more of SDF-1, SDF-1 receptor, IL-8, IL-8 receptor, IL-6 and IL-6 receptor encoded by the agent in the subject (i.e., gene therapy). In a non-limiting example, the agent may comprise, consist essentially of or consist of isolated cells (e.g., autologous, allogeneic or xenogeneic cells) transformed (e.g., transiently or stably transformed, preferably stably transformed) with said recombinant nucleic acid. In a non-limiting example, the agent may comprise, consist essentially of or consist of isolated cells (e.g., autologous, allogeneic or xenogeneic cells) naturally expressing or overexpressing said proteins. Administration of such cells to a subject shall effect expression of any one or more of SDF-1, SDF-1 receptor, IL-8, IL-8 receptor, IL-6 and IL-6 receptor by said cells in the subject (i.e., cell therapy).

Or the agent may be able to reduce the expression of said one or more nucleic acids or proteins selected from the group consisting of SDF-1, SDF-1 receptor, IL-8, IL-8 receptor, IL-6 and IL-6 receptor. For example, such agent may be selected from the group consisting of an antisense agent, a ribozyme and an agent capable of causing RNA interference.

Or the agent may be able to increase the level and/or activity of said one or more proteins selected from the group consisting of SDF-1, SDF-1 receptor, IL-8, IL-8 receptor, IL-6 and IL-6 receptor. For example, such agent may suitably comprise, consist essentially of or consist of SDF-1, SDF-1 receptor, IL-8, IL-8 receptor, IL-6 and/or IL-6 receptor protein, such as preferably isolated or recombinant SDF-1, SDF-1 receptor, IL-8, IL-8 receptor, IL-6 and/or IL-6 receptor protein; or the agent may suitably comprise, consist essentially of or consist of an agonist of SDF-1, IL-8 and/or IL-6 protein or of an agonist of SDF-1 receptor, IL-8 receptor and/or IL-6 receptor. Such agonist may be without limitation selected from a group consisting of an antibody or a fragment or derivative thereof, a protein or polypeptide, a peptide, a peptidomimetic, an aptamer, a photoaptamer, a nucleic acid, or a chemical substance, preferably an organic molecule, more preferably a small organic molecule. Where an agonist is an expressible molecule such as an antibody or a fragment or derivative thereof, a protein or polypeptide, a peptide or a nucleic acid, the agonist may be introduced to a subject or may be introduced by means of a recombinant nucleic acid comprising a sequence encoding the agonist operably linked to one or more regulatory sequences allowing for expression of said sequence encoding the agonist (e.g., gene therapy or cell therapy, supra).

Or the agent may be able to reduce the level and/or activity of said one or more proteins selected from the group consisting of SDF-1, SDF-1 receptor, IL-8, IL-8 receptor, IL-6 and IL-6 receptor. For example, the agent may suitably comprise, consist essentially of or consist of an antagonist of SDF-1, IL-8 and/or IL-6 protein or of an antagonist of SDF-1 receptor, IL-8 receptor and/or IL-6 receptor. Such antagonist may be without limitation selected from a group consisting of an antibody or a fragment or derivative thereof, a protein or polypeptide, a peptide, a peptidomimetic, an aptamer, a photoaptamer, a nucleic acid, or a chemical substance, preferably an organic molecule, more preferably a small organic molecule. Where an antagonist is an expressible molecule such as an antibody or a fragment or derivative thereof, a protein or polypeptide, a peptide or a nucleic acid, the antagonist may be introduced to a subject or may be introduced by means of a recombinant nucleic acid comprising a sequence encoding the antagonist operably linked to one or more regulatory sequences allowing for expression of said sequence encoding the antagonist (e.g., gene therapy or cell therapy, supra). For example, an antagonist may also encompass a deletion form of SDF-1, SDF-1 receptor, IL-8, IL-8 receptor, IL-6 or IL-6 receptor having a dominant negative activity over the respective native proteins.

By means of example and without limitation, agents which increase the level and/or activity of SDF-1, more preferably of human SDF-1 (SDF-1 agonists), may encompass (may be selected from the group comprising, consisting essentially of or consisting of) protein or peptide agonists such as, e.g., isolated or recombinant (preferably native human) SDF-1α, SDF-1β, SDF-1δ, SDF-1φ and/or SDF-1ε, CTCE-0214 (an SDF-1 analogue in which the C-terminus of SDF-1α is connected to the N-terminal region by a short bi-functional linker) (Perez et al. Exp. Hematol., 2004, vol. 32(3), 300-307). Sumo-SDF-1(S4V) (a protease-resistant SDF-1 resistant to matrix metalloproteinase-2 and exopeptidase cleavage and providing a long lasting effect (e.g., Segers et al. Circulation, 2007, vol. 116(15), 1683-1692)), SDF-1 overexpressing cells such as adeno-SDF1-infected mesenchymal or osteoblastic cell lines (Zhang et al. FASEB J, 2007, vol. 21(12), 3197-3207; Tang et al. Eur J Cardiothorac. Surg., 2009, vol. 36(4), 644-650), RNA agents such as miR-430 (miRNA shown to regulate SDF1-α and CXCR-7 mRNAs (Staton et al. Nat. Genet., 2011, vol. 43(3), 204-211)).

By means of example and without limitation, agents which reduce the level and/or activity of SDF-1, more preferably of human SDF-1 (SDF-1 antagonists), may encompass (may be selected from the group comprising, consisting essentially of or consisting of) RNA oligonucleotides such as NOX-A12 (Duda et al. Clin. Cancer Res., February 2011).

By means of example and without limitation, agents which increase the level and/or activity of SDF-1 receptor, more preferably of human SDF-1 receptor (SDF-1 receptor agonists), may encompass (may be selected from the group comprising, consisting essentially of or consisting of) protein or peptide agonists such as, e.g., isolated or recombinant (preferably native human) CXCR4 and/or CXCR7, or CXCR-4 overexpressing cells such as Adeno-CXCR4-infected mesenchymal or osteoblastic cell lines (Zhang et al. J. Mol. Cell. Cardiol., vol. 44(2), 281-292).

By means of example and without limitation, agents which reduce the level and/or activity of SDF-1 receptor, more preferably of human SDF-1 receptor (SDF-1 receptor antagonists), may encompass (may be selected from the group comprising, consisting essentially of or consisting of) peptides such as ATI-2341 or pepducin which is a CXCR4 antagonist (Tchernychev et al. PNAS, 2010, vol. 107(51), 22255-22259), and non-peptidic molecules, such as Plerixafor or AMD3100 (trade name Mozobil, Genzyme Inc.) which is a CXCR4 antagonist bicyclam in which the two cyclam rings are tethered by an aromatic bridge (Teicher, Biochem Pharmacol, 2011, vol. 81(1), 6-12), or the CXCR-7 blocker CCX2066 (ChemoCentryx Inc.) (Duda et al. Clin. Cancer Res., February 2011).

By means of example and without limitation, agents which increase the level and/or activity of IL-8, more preferably of human IL-8 (IL-8 agonists), may encompass (may be selected from the group comprising, consisting essentially of or consisting of) protein or peptide agonists such as, e.g., isolated or recombinant (preferably native human) IL-8 (CXCL-8) (e.g., human recombinant IL-8, such as a 78 amino acid IL-8 peptide purified from Escherichia coli as described by Lindley et al. PNAS, 1988, vol. 85(23), 9199-9203), granulocyte chemotactic protein (GCP-1), leukocyte cell derived chemotaxin (LECT), lymphocyte-derived neutrophil-activating factor (LYNAP), monocyte-derived neutrophil chemotactic factor (MDNCF), monocyte-derived neutrophil-activating peptide (MONAP), neutrophil activating factor (NAF) or neutrophil-activating peptide 1 (NAP-1).

By means of example and without limitation, agents which increase the level and/or activity of IL-8 receptor, more preferably of human IL-8 receptor (IL-8 receptor agonists), may encompass (may be selected from the group comprising, consisting essentially of or consisting of) protein or peptide agonists such as, e.g., isolated or recombinant (preferably native human) CXCR1 or CXCR2.

By means of example and without limitation, agents which reduce the level and/or activity of IL-8 receptor, more preferably of human IL-8 receptor (IL-8 receptor antagonists), may encompass (may be selected from the group comprising, consisting essentially of or consisting of) non-peptidic molecules such as Repertaxin or R(−)-2-(4-isobutylphenyl)propionyl methansulphonamide), L-lysin salt (Casilli et al. Biochem. Pharmacol., 2005, vol. 69(3), 385-394; Teicher, Biochem Pharmacol, 2011, vol. 81(1), 6-12), SCH-479833 or SCH-527123 (Singh et al. Clin. Cancer. Res., 2009, vol. 15(7), 2380-2386; Teicher, Biochem Pharmacol, 2011, vol. 81(1), 6-12).

By means of example and without limitation, agents which increase the level and/or activity of IL-6, more preferably of human IL-6 (IL-6 agonists), may encompass (may be selected from the group comprising, consisting essentially of or consisting of) protein or peptide agonists such as, e.g., isolated or recombinant (preferably native human) IL-6 (e.g., human recombinant IL-6 as described by Rozen et al. Bone, 2007, vol. 41(3), 437-445), hepatocyte stimulating factor (HSF), hybridoma growth factor (HGF), T-cell differentiation factor (CDF), B cell stimulatory factor 2 (BSF2) or Interferon β2 (IFNB2).

By means of example and without limitation, agents which increase the level and/or activity of IL-6 receptor, more preferably of human IL-6 receptor (IL-6 receptor agonists), may encompass (may be selected from the group comprising, consisting essentially of or consisting of) protein or peptide agonists such as, e.g., isolated or recombinant (preferably native human) IL-6 receptor (CD126), e.g., recombinant IL-6 receptor as described by Rozen et al. Bone, 2007, vol. 41(3), 437-445).

By means of example and without limitation, agents which reduce the level and/or activity of IL-6 receptor, more preferably of human IL-6 receptor (IL-6 receptor antagonists), may encompass (may be selected from the group comprising, consisting essentially of or consisting of) monoclonal antibodies such as Tocilizumab® (Roche) (Hennigan & Kavanaugh. Ther. Clin. Risk. Manag, 2008, vol. 4(4), 767-775; Kato et al. Exp. Mol. Pathol., 2008, vol. 84(3), 262-270).

The impaired bone fracture healing may be selected from the group consisting of mal-union fracture, delayed union fracture and non-union fracture.

In the screening assays as set forth above, modulation of the level and/or activity of said one or more nucleic acids or proteins selected from the group consisting of SDF-1, IL-8 and IL-6 by test agents may be advantageously tested by contacting (i.e., combining, exposing or incubating) said one or more nucleic acids or proteins with a test agent under conditions generally conducive for such modulation. By means of example and not limitation, where modulation of the activity and/or level of said one or more nucleic acids or proteins results from binding of the test agent to said one or more nucleic acids or proteins, said conditions may be generally conducive for such binding. For example and without limitation, modulation of the activity and/or level of said one or more nucleic acids or proteins by the test agent may be suitably tested in vitro; or may be tested in host cells or host organisms comprising said one or more nucleic acids or proteins and exposed to or configured to express the test agent.

In the screening assays as set forth above, binding between a test agent and said one or more nucleic acids or proteins selected from the group consisting of SDF-1, IL-8 and IL-6 may be advantageously tested by contacting (i.e., combining, exposing or incubating) said one or more nucleic acids or proteins with the test agent under conditions generally conducive for such binding. For example and without limitation, binding between the test agent and said one or more nucleic acids or proteins may be suitably tested in vitro; or may be tested in host cells or host organisms comprising said one or more nucleic acids or proteins and exposed to or configured to express the test agent.

Without limitation, the agents as intended throughout the specification may be capable of binding any one or more nucleic acids or proteins selected from the group consisting of SDF-1, IL-8 and IL-6 or of modulating the level and/or activity of any one or more nucleic acids or proteins selected from the group consisting of SDF-1, IL-8 and IL-6 in vitro, in a cell, in an organ and/or in an organism.

The above and further aspects and preferred embodiments of the invention are described in the following sections and in the appended claims. The subject matter of appended claims is hereby specifically incorporated in this specification.

### BRIEF DESCRIPTION OF FIGURES

FIG. 1 illustrates plasma levels of SDF-1 in an experiment comparing a group of non-union patients (NU) with healthy controls (HV), (A) all samples (HV, n=49; NU, n=15), (B) samples where plasma is collected in heparin tubes (HV, n=26; NU, n=11), (C) samples where plasma is collected in EDTA tubes (HV, n=40; NU, n=5).

FIG. 2 illustrates serum levels of IL-8 in an experiment comparing a group of non-union patients (NU, n=4) with healthy controls (HV, n=18).

FIG. 3 illustrates serum levels of IL-6 in an experiment comparing a group of non-union patients (NU; n=13) with healthy controls (HV; n=29).

FIG. 4A illustrates levels of SDF-1 in supernatant of osteoblastic cell (OB) culture comparing a group of non-union patients (NU, n=6) with healthy controls (HV, n=9).

FIG. 4B illustrates levels of SDF-1 in supernatant of mesenchymal cell (MSC) culture comparing a group of non-union patients (NU, n=6) with healthy controls (HV, n=9).

FIG. 5 illustrates levels of IL-6 in supernatant of osteoblastic cell (OB) culture comparing a group of non-union patients (NU, n=6) with healthy controls (HV, n=10).

FIG. 6 represents photographs illustrating the results of bone formation in a calvarial model in mice for the negative control (vehicle, PBS-HSA); (A) Imaging (X-ray) of calvarial defect, (B) Histological analysis with Hematoxylin-eosin at 20 times magnification made on coronal section of (1) calvarial bone defect and (2) normal bone, (C) Histological analysis with Masson's trichrome at 20 times magnification made on coronal section of (1) calvarial bone defect and (2) normal bone.

FIG. 7 represents photographs illustrating the results of bone formation in a calvarial model in mice for the positive control (BMP-2, 5 μg); (A) Imaging (X-ray) of calvarial defect, (B) Histological analysis made on coronal section with Hematoxylin-eosin at 20 times magnification, (C) Histological analysis made on coronal section with Masson's trichrome at 20 times magnification. Boxes and arrows indicate the zones where bone formation was observed.

FIG. 8 represents photographs illustrating the results of bone formation in a calvarial model in mice treated with a composition comprising the IL-8 peptide having the amino acid sequence of SEQ ID No. 1; (A) Imaging (X-ray) of calvarial defect, (B) Histological analysis made on coronal section with Hematoxylin-eosin at 20 times (left) and 40 times (rights) magnification, (C) Histological analysis made on coronal section with Masson's trichrome at 20 times (left) and 40 times (rights) magnification, (D) Histological analysis made on coronal section with Safranin-O at 20 times (left) and 40 times (rights) magnification. Boxes and arrows indicate the zones where bone formation was observed. (1) New bone formation; (2) hypertrophic chondrocytes.

FIG. 9 represents photographs illustrating the results of bone formation in a calvarial model in mice treated with a composition comprising the IL-8 peptide having the amino acid sequence of SEQ ID No. 2; (A) Imaging (X-ray) of calvarial defect, (B) Histological analysis made on two coronal sections of the calvarial defect with Hematoxylin-eosin at 20 times (left) and 40 times (rights) magnification, (C) Histological analysis made on coronal section with Masson's trichrome at 20 times (left) and 40 times (rights) magnification, (D) Histological analysis made on coronal section with Safranin-O at 20 times (left) and 40 times (rights) magnification. Boxes and arrows indicate the zones where bone formation was observed. (1) Hypertrophic chondrocytes.

### DETAILED DESCRIPTION OF THE INVENTION

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The term also encompasses “consisting of” and “consisting essentially of”.

The recitation of numerical ranges by endpoints includes all numbers and fractions subsumed within the respective ranges, as well as the recited endpoints.

The term “about” as used herein when referring to a measurable value such as a parameter, an amount, a temporal duration, and the like, is meant to encompass variations of and from the specified value, in particular variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” refers is itself also specifically, and preferably, disclosed.

Whereas the term “one or more”, such as one or more members of a group of members, is clear per se, by means of further exemplification, the term encompasses inter alia a reference to any one of said members, or to any two or more of said members, such as, e.g., any ≧3, ≧4, ≧5, ≧6 or ≧7 etc. of said members, and up to all said members.

All documents cited in the present specification are hereby incorporated by reference in their entirety.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

For general methods relating to the invention, reference is made inter alia to well-known textbooks, including, e.g., “Molecular Cloning: A Laboratory Manual, 2nd Ed.” (Sambrook et al., 1989), Animal Cell Culture (R. I. Freshney, ed., 1987), the series Methods in Enzymology (Academic Press), Gene Transfer Vectors for Mammalian Cells (J. M. Miller & M. P. Calos, eds., 1987); “Current Protocols in Molecular Biology and Short Protocols in Molecular Biology, 3rd Ed.” (F. M. Ausubel et al., eds., 1987 & 1995); Recombinant DNA Methodology II (R. Wu ed., Academic Press 1995).

General techniques in cell culture and media uses are outlined inter alia in Large Scale Mammalian Cell Culture (Hu et al. 1997. Curr Opin Biotechnol 8: 148); Serum-free Media (K. Kitano. 1991. Biotechnology 17: 73); or Large Scale Mammalian Cell Culture (Curr Opin Biotechnol 2: 375, 1991).

The term “protein” as used herein generally encompasses macromolecules comprising one or more polypeptide chains, i.e., polymeric chains of amino acid residues linked by peptide bonds. The term may encompass naturally, recombinantly, semi-synthetically or synthetically produced proteins. The term also encompasses proteins that carry one or more co- or post-expression modifications of the polypeptide chain(s), such as, without limitation, glycosylation, acetylation, phosphorylation, sulfonation, methylation, ubiquitination, signal peptide removal, N-terminal Met removal, conversion of pro-enzymes or pre-hormones into active forms, etc. The term further also includes protein variants or mutants which carry amino acid sequence variations vis-à-vis a corresponding native protein, such as, e.g., amino acid deletions, additions and/or substitutions. The term contemplates both full-length proteins and protein parts or fragments, e.g., naturally-occurring protein parts that ensue from processing of such full-length proteins.

The term “nucleic acid” as used herein generally encompasses polymers of any length composed essentially of nucleotides, e.g., deoxyribonucleotides and/or ribonucleotides. Nucleic acids can comprise purine and/or pyrimidine bases and/or other natural (e.g., xanthine, inosine, hypoxanthine), chemically or biochemically modified (e.g., methylated), non-natural, or derivatised nucleotide bases. The backbone of nucleic acids can comprise sugars and phosphate groups, as can typically be found in RNA or DNA, and/or one or more modified or substituted sugars (such as, e.g., 2′-O-alkylated, e.g., 2′-O-methylated or 2′-O-ethylated; or 2′-O,4′-C-alkynelated, e.g., 2′-O,4′-C-ethylated sugars) and/or one or more modified or substituted phosphate groups (e.g., phosphodiester, phosphorothioate, phosphorodithioate, methylphosphonate, phosphoramidate, alkyl phosphotriester, sulfamate, 3′-thioacetal, methylene (methylimino), 3′-N-carbamate, morpholino carbamate, and peptide nucleic acids (PNAs)). The term “nucleic acid” further preferably encompasses DNA, RNA and DNA/RNA hybrid molecules, specifically including hnRNA, pre-mRNA, mRNA, cDNA, genomic DNA, amplification products, oligonucleotides, and synthetic (e.g. chemically synthesised) DNA, RNA or DNA/RNA hybrids. A nucleic acid can be naturally occurring, e.g., present in or isolated from nature, can be recombinant, i.e., produced by recombinant DNA technology, and/or can be, partly or entirely, chemically or biochemically synthesised. A “nucleic acid” can be double-stranded, partly double stranded, or single-stranded. Where single-stranded, the nucleic acid can be the sense strand or the antisense strand. In addition, nucleic acid can be circular or linear.

The term “isolated” with reference to a particular component (such as for instance a nucleic acid, protein, polypeptide or peptide) generally denotes that such component exists in separation from—for example, has been separated from or prepared and/or maintained in separation from—one or more other components of its natural environment. For instance, an isolated human or animal protein or complex may exist in separation from a human or animal body where it naturally occurs.

The term “isolated” as used herein may preferably also encompass the qualifier “purified”. By means of example, the term “purified” with reference to nucleic acids, proteins, polypeptides or peptides does not require absolute purity. Instead, it denotes that such nucleic acids, proteins, polypeptides or peptides are in a discrete environment in which their abundance (conveniently expressed in terms of mass or weight or concentration) relative to other nucleic acids, proteins, polypeptides or peptides is greater than in a biological sample. A discrete environment denotes a single medium, such as for example a single solution, gel, precipitate, lyophilisate, etc. Purified nucleic acids, proteins, polypeptides or peptides may be obtained by known methods including, for example, laboratory or recombinant synthesis, chromatography, preparative electrophoresis, centrifugation, precipitation, affinity purification, etc.

The inventors identified stromal derived factor-1 (SDF-1 or CXCL12), SDF-1 receptor, interleukin-8 (IL-8 or CXCL8), IL-8 receptor, interleukin-6 (IL-6) or IL-6 receptor as valuable targets for therapeutic and/or prophylactic interventions in impaired fracture healing. The inventors thus contemplate these molecules as useful targets for therapeutic and/or prophylactic interventions in the treatment of any fractures.

The terms “non-union fracture”, “fracture non-union”, “non-union” or “NU” interchangeably concern a fracture which due to various factors fails to heal in a normal time period. NU includes inter alia tight non-unions and unstable non-unions or pseudarthrosis. The terms “mal-union fracture”, “fracture mal-union” or “mal-union” interchangeably concern an imperfect union of previously fragmented bone. The terms “delayed union fracture” or “delayed union” interchangeably relate to a fracture in which healing has not occurred in the expected time and the outcome remains uncertain. Non-union, mal-union and delayed union fractures are encompassed herein by the term “impaired bone fracture healing” or “impaired fracture healing”. Impaired fracture healing hence requires some form of intervention to stimulate healing.

The time period at which impaired fracture healing is concluded in practice varies depending on the particular fracture, but it is generally accepted that a fracture not healed by 6 months post injury will not heal without intervention. It has also been suggested to conclude that impaired fracture healing will result if a fracture shows no sign of progressing towards healing by 3 months post injury, or simply if a fracture has not healed in the time an experienced fracture surgeon would expect it to heal.

Reference throughout this specification to diseases or conditions encompasses any such diseases or conditions as disclosed herein insofar consistent with the context of a particular recitation, more specifically encompasses impaired fracture healing. Reference herein to the treatment of a fracture may encompass the treatment of impaired fracture healing.

As used herein, the reference to any one nucleic acid or protein corresponds to the nucleic acid, protein, polypeptide or peptide commonly known under the respective designations in the art. The terms encompass such nucleic acids, proteins, polypeptides or peptides of any organism where found, and particularly of animals, preferably warm-blooded animals, more preferably vertebrates, yet more preferably mammals, including humans and non-human mammals, still more preferably of humans. The terms particularly encompass such nucleic acids, proteins, polypeptides or peptides with a native sequence, i.e., ones of which the primary sequence is the same as that of the nucleic acids, proteins, polypeptides or peptides found in or derived from nature. A skilled person understands that native sequences may differ between different species due to genetic divergence between such species. Moreover, native sequences may differ between or within different individuals of the same species due to normal genetic diversity (variation) within a given species. Also, native sequences may differ between or even within different individuals of the same species due to post-transcriptional or post-translational modifications. Any such variants or isoforms of nucleic acids, proteins, polypeptides or peptides are intended herein. Accordingly, all sequences of nucleic acids, proteins, polypeptides or peptides found in or derived from nature are considered “native”. The terms encompass the nucleic acids, proteins, polypeptides or peptides when forming a part of a living organism, organ, tissue or cell, when forming a part of a biological sample, as well as when at least partly isolated from such sources. The terms also encompass the nucleic acids, proteins, polypeptides or peptides when produced by recombinant or synthetic means.

Exemplary human nucleic acids, proteins, polypeptides or peptides as taught herein may be as annotated under NCBI Genbank (http://www.ncbi.nlm.nih.gov/) accession numbers given below. A skilled person can also appreciate that in some instances said sequences may be of precursors (e.g., preproteins) of the nucleic acids, proteins, polypeptides or peptides as taught herein and may include parts which are processed away from the mature nucleic acids, proteins, polypeptides or peptides. A skilled person can further appreciate that although only one or more isoforms may be listed below, all isoforms are intended. Unless otherwise specified, the entries below are presented in the form: Name (Code; Genbank accession number for one or more representative mRNA sequences (e.g., isoforms), followed by a period and the Genbank sequence version; Genbank accession number for one or more corresponding representative amino acid sequences (e.g., isoforms), followed by a period and the Genbank sequence version):

Stromal derived factor-1 and isoforms α, β, γ, φ and ε (SDF-1 or CXCL12; NM_{—}199168.3, NM_{—}000609.5, NM_{—}001033886.2, NM_{—}001178134.1; NP_{—}954637.1, NP_{—}000600.1, NP_{—}001029058.1, NP_{—}001171605.1)

Interleukin-8 (IL-8, CXCL8, GCP-1, GCP1, LECT, LUCT, LYNAP, MDNCF, MONAP, NAF, NAP-1 or NAP 1; NM_{—}000584.3; NP_{—}000575.1)

Interleukin-6 (IL-6, HSF, HGF, CDF, BSF2 or IFNB2; NM_{—}000600.3, NP_{—}000591.1)

Chemokine (C-X-C motif) receptor 4 isoforms a and b (CXCR4, FB22, HM89, LAP3, LCR1, NPYR, WHIM, CD184, LESTR, NPY3R, NPYRL, HSY3RR, NPYY3R, D2S201E; NM_{—}001008540.1, NM_{—}003467.2, NP_{—}001008540.1, NP_{—}003458.1)

Chemokine (C-X-C motif) receptor 7 (CXCR7, RDC1, CMKOR1, GPR159, NM_{—}020311.2, NP_{—}064707.1)

Chemokine (C-X-C motif) receptor 1 (CXCR1, C-C, CD128, CD181, CKR-1, IL8R1, IL8RA, CMKAR1, IL8RBA, CDw128a, C-C-CKR-1, NM_{—}000634.2, NP_{—}000625.1)

Chemokine (C-X-C motif) receptor 2 transcript variants 1 and 2 (CXCR2, CD182, IL8R2, IL8RA, IL8RB, CMKAR2, CDw128b, NM_{—}001168298.1, NM_{—}001557.3, NP_{—}001161770.1, NP_{—}001548.1)

Interleukin 6 receptor isoforms 1 and 2 (CD126, IL6RA, IL-6R-1, MGC104991, IL-6R-alpha, IL6R, NM_{—}000565.2, NM_{—}181359.1, NP_{—}000556.1, NP_{—}852004.1)

Unless otherwise apparent from the context, reference herein to any nucleic acid, protein, polypeptide or peptide may generally also encompass modified forms of said nucleic acid, protein, polypeptide or peptide such as bearing post-expression or chemical modifications including, for example, phosphorylation, glycosylation, lipidation, methylation, cysteinylation, sulphonation, glutathionylation, acetylation, oxidation of methionine to methionine sulphoxide or methionine sulphone, and the like.

A nucleic acid, protein, polypeptide or peptide may be preferably human, i.e., their primary sequence may be the same as a corresponding primary sequence of or present in a naturally occurring human nucleic acid, protein, polypeptide or peptide. Hence, the qualifier “human” in this connection relates to the primary sequence of the respective nucleic acid, protein, polypeptide or peptide, rather than to its origin or source. For example, such nucleic acid, protein, polypeptide or peptide may be present in or isolated from samples of human subjects or may be obtained by other means (e.g., by recombinant expression, cell-free translation or non-biological peptide synthesis).

The reference herein to any nucleic acid, protein, polypeptide or peptide may also encompass fragments thereof. The term “fragment” of a nucleic acid generally refers to 5′- and/or 3′-terminally deleted or truncated forms of said nucleic acid. The term “fragment” of a protein, polypeptide or peptide generally refers to N-terminally and/or C-terminally deleted or truncated forms of said protein, polypeptide or peptide. Without limitation, a fragment of a nucleic acid, protein, polypeptide or peptide may represent at least about 5%, or at least about 10%, e.g., ≧20%, ≧30% or ≧40%, such as preferably ≧50%, e.g., ≧60%, ≧70% or ≧80%, or more preferably ≧90% or ≧95% of the nucleotide sequence of said nucleic acid or of the amino acid sequence of said protein, polypeptide or peptide.

The reference herein to any nucleic acid, protein, polypeptide or peptide may also encompass variants thereof. The term “variant” of a nucleic acid, protein, polypeptide or peptide refers to nucleic acids, proteins, polypeptides or peptides the sequence (i.e., nucleotide sequence or amino acid sequence, respectively) of which is substantially identical (i.e., largely but not wholly identical) to the sequence of said recited nucleic acid, protein or polypeptide, e.g., at least about 80% identical or at least about 85% identical, e.g., preferably at least about 90% identical, e.g., at least 91% identical, 92% identical, more preferably at least about 93% identical, e.g., at least 94% identical, even more preferably at least about 95% identical, e.g., at least 96% identical, yet more preferably at least about 97% identical, e.g., at least 98% identical, and most preferably at least 99% identical. Preferably, a variant may display such degrees of identity to a recited nucleic acid, protein, polypeptide or peptide when the whole sequence of the recited nucleic acid, protein, polypeptide or peptide is queried in the sequence alignment (i.e., overall sequence identity). Also included among fragments and variants of a nucleic acid, protein, polypeptide or peptide are fusion products of said nucleic acid, protein, polypeptide or peptide with another, usually unrelated, nucleic acid, protein, polypeptide or peptide.

Sequence identity may be determined using suitable algorithms for performing sequence alignments and determination of sequence identity as know per se. Exemplary but non-limiting algorithms include those based on the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Left 174: 247-250), for example using the published default settings or other suitable settings (such as, e.g., for the BLASTN algorithm: cost to open a gap=5, cost to extend a gap=2, penalty for a mismatch=−2, reward for a match=1, gap x_dropoff=50, expectation value=10.0, word size=28; or for the BLASTP algorithm: matrix=Blosum62, cost to open a gap=11, cost to extend a gap=1, expectation value=10.0, word size=3).

A variant of a nucleic acid, protein, polypeptide or peptide may be a homologue (e.g., orthologue or paralogue) of said nucleic acid, protein, polypeptide or peptide. As used herein, the term “homology” generally denotes structural similarity between two macromolecules, particularly between two nucleic acids, proteins or polypeptides, from same or different taxons, wherein said similarity is due to shared ancestry.

Where the present specification refers to or encompasses fragments and/or variants of nucleic acids, proteins, polypeptides or peptides, this preferably denotes variants and/or fragments which are “functional”, i.e., which at least partly retain the biological activity or intended functionality of the respective nucleic acids, proteins, polypeptides or peptides. By means of an example and not limitation, a functional fragment and/or variant of an antisense agent or RNAi agent shall at least partly retain the functionality of said agent, i.e., its ability to reduce or abolish the expression of a target molecule (gene). By means of another example and not limitation, a functional fragment and/or variant of an SDF-1, IL-8 or IL-6 nucleic acid, protein, polypeptide or peptide shall at least partly retain the biological activity of SDF-1, IL-8 or IL-6, respectively. For example, it may retain one or more aspects of the biological activity of SDF-1, IL-8 or IL-6, such as, e.g., ability to bind to one or more cognate receptors, to participate in one or more cellular pathways, etc. By means of another example and not limitation, a functional fragment and/or variant of an SDF-1 receptor, IL-8 receptor or IL-6 receptor nucleic acid, protein, polypeptide or peptide shall at least partly retain the biological activity of SDF-1 receptor, IL-8 receptor or IL-6 receptor, respectively. For example, it may retain one or more aspects of the biological activity of SDF-1 receptor, IL-8 receptor or IL-6 receptor, such as, e.g., ability to bind one or more cognate ligands, to effect cellular signalling when binding a ligand, etc. Preferably, a functional fragment and/or variant may retain at least about 20%, e.g., at least 30%, or at least about 40%, or at least about 50%, e.g., at least 60%, more preferably at least about 70%, e.g., at least 80%, yet more preferably at least about 85%, still more preferably at least about 90%, and most preferably at least about 95% or even about 100% or higher of the intended biological activity or functionality compared to the corresponding nucleic acid, protein, polypeptide or peptide.

The term “modulate” or “modulating” generally denotes a qualitative or quantitative alteration, change or variation specifically encompassing both increase (e.g., activation) or decrease (e.g., inhibition), of that which is being modulated. The term encompasses any extent of such modulation.

For example, where modulation effects a determinable or measurable variable, then modulation may encompass an increase in the value of said variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of said variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation.

Preferably, modulation of the activity and/or level of intended target(s) (particularly SDF-1, SDF-1 receptor, IL-8, IL-8 receptor, IL-6 or IL-6 receptor) may be specific or selective, i.e., the activity and/or level of intended target(s) may be modulated without substantially altering the activity and/or level of random, unrelated targets.

Reference to the “activity” of a target may generally encompass any one or more aspects of the biological activity of the target, such as without limitation any one or more aspects of its biochemical activity, enzymatic activity, signalling activity, interaction activity, ligand activity, receptor activity and/or structural activity, e.g., within a cell, tissue, organ or an organism. By means of an example and not limitation, reference to the activity of SDF-1, IL-8 or IL-6 may particularly denote their activity as a ligand, i.e., their ability to bind to one or more cognate receptors, and/or their activity as a signalling molecule, i.e., their ability to participate in one or more cellular signalling pathways, etc. By means of another example and not limitation, reference to the activity of SDF-1 receptor, IL-8 receptor or IL-6 receptor may particularly denote their activity as a receptor, i.e., their ability to bind one or more cognate ligands and to effect downstream cellular signalling when bound by the ligand, etc.

Reference to the “level” of a target may preferably encompass the quantity and/or the availability (e.g., availability for performing its biological activity) of the target, e.g., within a cell, tissue, organ or an organism.

Except when noted, “subject” or “patient” are used interchangeably and refer to animals, preferably warm-blooded animals, more preferably vertebrates, even more preferably mammals, still more preferably primates, and specifically includes human patients and non-human mammals and primates. Preferred patients are human subjects.

As used herein, a phrase such as “a subject in need of treatment” includes subjects that would benefit from treatment of a given condition, particularly impaired bone healing. Such subjects may include, without limitation, those that have been diagnosed with said condition, those prone to contract or develop said condition and/or those in whom said condition is to be prevented.

The terms “treat” or “treatment” encompass both the therapeutic treatment of an already developed disease or condition, such as the therapy of an already developed impaired bone healing, as well as prophylactic or preventative measures, wherein the aim is to prevent or lessen the chances of incidence of an undesired affliction, such as to prevent the chances of contraction and progression of impaired bone healing. Beneficial or desired clinical results may include, without limitation, alleviation of one or more symptoms or one or more biological markers, diminishment of extent of disease, stabilised (i.e., not worsening) state of disease, delay or slowing of disease progression, amelioration or palliation of the disease state, and the like. “Treatment” can also mean prolonging survival as compared to expected survival if not receiving treatment.

The term “prophylactically effective amount” refers to an amount of an active compound or pharmaceutical agent that inhibits or delays in a subject the onset of a disorder as being sought by a researcher, veterinarian, medical doctor or other clinician. The term “therapeutically effective amount” as used herein, refers to an amount of active compound or pharmaceutical agent that elicits the biological or medicinal response in a subject that is being sought by a researcher, veterinarian, medical doctor or other clinician, which may include inter alia alleviation of the symptoms of the disease or condition being treated. Methods are known in the art for determining therapeutically and prophylactically effective doses for the present agents.

As used herein, the term “agent” broadly refers to any chemical (e.g., inorganic or organic), biochemical or biological substance, molecule or macromolecule (e.g., biological macromolecule), a combination or mixture thereof, a sample of undetermined composition, or an extract made from biological materials such as bacteria, plants, fungi, or animal cells or tissues. Preferred though non-limiting “agents” include nucleic acids, oligonucleotides, ribozymes, polypeptides or proteins, peptides, peptidomimetics, antibodies and fragments and derivatives thereof, aptamers, photoaptamers, chemical substances, preferably organic molecules, more preferably small organic molecules, lipids, carbohydrates, polysaccharides, etc., and any combinations thereof.

As taught herein, an agent may for example specifically bind to a target. The term “specifically bind” as used throughout this specification means that an agent binds to one or more desired targets, such as to one or more desired nucleic acids, proteins, polypeptides or peptides substantially to the exclusion of other molecules which are random or unrelated, and optionally substantially to the exclusion of other molecules that are structurally related. Binding of an agent to a target may be evaluated inter alia using conventional interaction-querying methods, such as co-immunoprecipitation, immunoassay methods, chromatography methods, gel elecrophoresis methods, yeast two hybrid methods, or combinations thereof.

The term “specifically bind” does not necessarily require that an agent binds exclusively to its intended target(s). For example, an agent may be said to specifically bind to the desired nucleic acid(s), protein(s), polypeptide(s) or peptide(s) if its affinity for such intended target(s) under the conditions of binding is at least about 2-fold greater, preferably at least about 5-fold greater, more preferably at least about 10-fold greater, yet more preferably at least about 25-fold greater, still more preferably at least about 50-fold greater, and even more preferably at least about 100-fold or more greater, than its affinity for a non-target molecule.

Preferably, the agent may bind to its intended target(s) with affinity constant (K_{A}) of such binding K_{A}≧1×10⁶ M⁻¹, more preferably K_{A}≧1×10⁷ M⁻¹, yet more preferably K_{A}≧1×10⁸ M⁻¹, even more preferably K_{A}≧1×10⁹ M⁻¹, and still more preferably K_{A}≧1×10¹⁰ M⁻¹ or K_{A}≧1×10¹¹ M⁻¹, wherein K_{A}=[A_T]/[A][T], A denotes the agent, T denotes the intended target. Determination of K_{A} can be carried out by methods known in the art, such as for example, using equilibrium dialysis and Scatchard plot analysis.

Certain types of agents comprised in this specification are described in the following in more detail.

As used herein, the term “antibody” is used in its broadest sense and generally refers to any immunologic binding agent. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro, in cell culture, or in vivo.

In an embodiment, an antibody may be any of IgA, IgD, IgE, IgG and IgM classes, and preferably IgG class antibody.

In an embodiment, the antibody may be a polyclonal antibody, e.g., an antiserum or immunoglobulins purified there from (e.g., affinity-purified).

In another preferred embodiment, the antibody may be a monoclonal antibody or a mixture of monoclonal antibodies. Monoclonal antibodies can target a particular antigen or a particular epitope within an antigen with greater selectivity and reproducibility.

By means of example and not limitation, monoclonal antibodies may be made by the hybridoma method first described by Kohler et al. 1975 (Nature 256: 495), or may be made by recombinant DNA methods (e.g., as in U.S. Pat. No. 4,816,567). Monoclonal antibodies may also be isolated from phage antibody libraries using techniques as described by Clackson et al. 1991 (Nature 352: 624-628) and Marks et al. 1991 (J Mol Biol 222: 581-597), for example.

In further embodiments, antibody agents may be antibody fragments. “Antibody fragments” comprise a portion of an intact antibody, comprising the antigen-binding or variable region thereof. Examples of antibody fragments include Fab, Fab′, F(ab′)2, Fv and scFv fragments; diabodies; linear antibodies; single-chain antibody molecules; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius) also including camel heavy-chain antibodies V_{H}H, llama (e.g., Lama paccos, Lama glama or Lama vicugna) also including llama heavy-chain antibodies V_{H}H, or horse.

A skilled person will understand that an antibody can include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

Methods of producing polyclonal and monoclonal antibodies as well as fragments thereof are well known in the art, as are methods to produce recombinant antibodies or fragments thereof (see for example, Harlow and Lane, “Antibodies: A Laboratory Manual”, Cold Spring Harbour Laboratory, New York, 1988; Harlow and Lane, “Using Antibodies: A Laboratory Manual”, Cold Spring Harbour Laboratory, New York, 1999, ISBN 0879695447; “Monoclonal Antibodies: A Manual of Techniques”, by Zola, ed., CRC Press 1987, ISBN 0849364760; “Monoclonal Antibodies: A Practical Approach”, by Dean & Shepherd, eds., Oxford University Press 2000, ISBN 0199637229; Methods in Molecular Biology, vol. 248: “Antibody Engineering: Methods and Protocols”, Lo, ed., Humana Press 2004, ISBN 1588290921).

Methods for immunising animals, e.g., non-human animals such as laboratory or farm animals, using immunising antigens (such as, e.g., the herein disclosed complexes) optionally fused to or covalently or non-covalently linked, bound or adsorbed to a presenting carrier, and preparation of antibody or cell reagents from immune sera is well-known per se and described in documents referred to elsewhere in this specification. The animals to be immunised may include any animal species, preferably warm-blooded species, more preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel, llama or horse. The term “presenting carrier” or “carrier” generally denotes an immunogenic molecule which, when bound to a second molecule, augments immune responses to the latter, usually through the provision of additional T cell epitopes. The presenting carrier may be a (poly)peptidic structure or a non-peptidic structure, such as inter alia glycans, polyethylene glycols, peptide mimetics, synthetic polymers, etc. Exemplary non-limiting carriers include human Hepatitis B virus core protein, multiple C3d domains, tetanus toxin fragment C or yeast Ty particles.

Selection of agents specifically binding to one or more targets of interest to the exclusion of other molecules (non-targets) may suitably involve methods for subtracting or removing from agents that bind to said one or more targets those agents that also cross-react or cross-bind with one or more non-targets. Such subtraction may be readily performed as known in the art by a variety of affinity separation methods, such as affinity chromatography, affinity solid phase extraction, affinity magnetic extraction, etc.

The term “aptamer” refers to single-stranded or double-stranded oligo-DNA, oligo-RNA or oligo-DNA/RNA or any analogue thereof, that can specifically bind to a target molecule. Advantageously, aptamers can display fairly high specificity and affinity (e.g., K_{A} in the order 1×10⁹ M⁻¹) for their targets. Aptamer production is described inter alia in U.S. Pat. No. 5,270,163; Ellington & Szostak 1990 (Nature 346: 818-822); Tuerk & Gold 1990 (Science 249: 505-510); or “The Aptamer Handbook: Functional Oligonucleotides and Their Applications”, by Klussmann, ed., Wiley-VCH 2006, ISBN 3527310592, incorporated by reference herein. The term “photoaptamer” refers to an aptamer that contains one or more photoreactive functional groups that can covalently bind to or crosslink with a target molecule.

The term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol 13: 132-134).

The term “small molecule” refers to compounds, preferably organic compounds, with a size comparable to those organic molecules generally used in pharmaceuticals. The term excludes biological macromolecules (e.g., proteins, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da.

The term “antisense” generally refers to an agent (e.g., an oligonucleotide) configured to specifically anneal with (hybridise to) a given sequence in a target nucleic acid, such as for example in a target DNA, hnRNA, pre-mRNA or mRNA, and typically comprises, consist essentially of or consist of a nucleic acid sequence that is complementary or substantially complementary to said target nucleic acid sequence. Antisense agents suitable for use herein may typically be capable of annealing with (hybridising to) the respective target nucleic acid sequences at high stringency conditions, and capable of hybridising specifically to the target under physiological conditions.

The terms “complementary” or “complementarity” as used herein with reference to nucleic acids, refer to the normal binding of single-stranded nucleic acids under permissive salt (ionic strength) and temperature conditions by base pairing, preferably Watson-Crick base pairing. By means of example, complementary Watson-Crick base pairing occurs between the bases A and T, A and U or G and C. For example, the sequence 5′-A-G-U-3′ is complementary to sequence 5′-A-C-U-3′.

The term “ribozyme” generally refers to a nucleic acid molecule, preferably an oligonucleotide or oligonucleotide analogue, capable of catalytically cleaving a polynucleotide. Preferably, a “ribozyme” may be capable of cleaving mRNA of a given target protein, thereby reducing translation thereof. Exemplary ribozymes contemplated herein include, without limitation, hammer head type ribozymes, ribozymes of the hairpin type, delta type ribozymes, etc. For teaching on ribozymes and design thereof, see, e.g., U.S. Pat. No. 5,354,855, U.S. Pat. No. 5,591,610, Pierce et al. 1998 (Nucleic Acids Res 26: 5093-5101), Lieber et al. 1995 (Mol Cell Biol 15: 540-551), and Benseler et al. 1993 (J Am Chem Soc 115: 8483-8484).

“RNA interference” or “RNAi” technology is known in the art, and refers generally to the process and means of sequence-specific post-transcriptional gene silencing mediated particularly by short interfering nucleic acids (siNA). For teaching on RNAi molecules and design thereof, see inter alia Elbashir et al. 2001 (Nature 411: 494-501), Reynolds et al. 2004 (Nat Biotechnol 22: 326-30), http://rnaidesigner.invitrogen.com/rnaiexpress, Wang & Mu 2004 (Bioinformatics 20: 1818-20), Yuan et al. 2004 (Nucleic Acids Res 32(Web Server issue): W130-4), by M Sohail 2004 (“Gene Silencing by RNA Interference: Technology and Application”, 1^{st} ed., CRC, ISBN 0849321417), U Schepers 2005 (“RNA Interference in Practice: Principles, Basics, and Methods for Gene Silencing in C. elegans, Drosophila, and Mammals”, 1^{st} ed., Wiley-VCH, ISBN 3527310207), and DR Engelke & JJ Rossi 2005 (“Methods in Enzymology, Volume 392: RNA Interference”, 1^{st} ed., Academic Press, ISBN 0121827976).

An RNAi agent typically comprises, consists essentially of or consists of a double-stranded portion or region (notwithstanding the optional and potentially preferred presence of single-stranded overhangs) of annealed complementary strands, one of which has a sequence corresponding to a target nucleotide sequence (hence, to at least a portion of an mRNA) of the target gene to be down-regulated. The other strand of the RNAi agent is complementary to said target nucleotide sequence.

Whereas the sequence of an RNAi agent need not be completely identical to a target sequence to be down-regulated, the number of mismatches between a target sequence and a nucleotide sequence of the RNAi agent is preferably no more than 1 in 5 bases, or 1 in 10 bases, or 1 in 20 bases, or 1 in 50 bases.

Preferably, to ensure specificity of RNAi agents towards the desired targets over unrelated molecules, the sequence of said RNAi agents may be at least about 80% identical, preferably at least about 90% identical, more preferably at least about 95% identical, such as, e.g., about 96%, about 97%, about 98%, about 99% and up to 100% identical to the respective target sequence.

An RNAi agent may be formed by separate sense and antisense strands or, alternatively, by a common strand providing for fold-back stem-loop or hairpin design where the two annealed strands of an RNAi agent are covalently linked.

An siRNA molecule may be typically produced, e.g., synthesised, as a double stranded molecule of separate, substantially complementary strands, wherein each strand is about 18 to about 35 bases long, preferably about 19 to about 30 bases, more preferably about 20 to about 25 bases and even more preferably about 21 to about 23 bases.

shRNA is in the form of a hairpin structure. shRNA can be synthesized exogenously or can be formed by transcribing from RNA polymerase III promoters in vivo. Preferably, shRNAs can be engineered in host cells or organisms to ensure continuous and stable suppression of a desired gene. It is known that siRNA can be produced by processing a hairpin RNA in cells.

RNAi agents as intended herein may include any modifications as set out elsewhere in this specification for nucleic acids and oligonucleotides, in order to improve their therapeutic properties.

At least one strand of an RNAi molecules may have a 3′ overhang from about 1 to about 6 bases in length, e.g., from 2 to 4 bases, more preferably from 1 to 3 bases. For example, one strand may have a 3′ overhang and the other strand may be either blunt-ended or may also have a 3′ overhang. The length of the overhangs may be the same or different for each strand. The 3′ overhangs can be stabilised against degradation. For example, the RNA may be stabilised by including purine nucleotides, such as A or G nucleotides. Alternatively, substitution of pyrimidine nucleotides by modified analogues, e.g., substitution of U 3′ overhangs by 2′-deoxythymidine is tolerated and does not affect the efficiency of RNAi.

An exemplary but non-limiting siRNA molecule may be characterized by any one or more, and preferably by all of the following criteria:
at least about 80% sequence identity, more preferably at least about 90% or at least about 95% or at least about 97% sequence identity to target mRNA;
having a sequence which targets an area of the target gene present in mature mRNA (e.g., an exon or alternatively spliced intron);
showing a preference for targeting the 3′ end of the target gene.

The exemplary siRNA may be further characterised by one or more or all of the following criteria:
having a double-stranded nucleic acid length of between 16 to 30 bases and preferably of between 18 to 23 bases, and preferably of 19 nucleotides;
having GC content between about 30 and about 50%
having a TT(T) sequence at 3′ end;
showing no secondary structure when adopting the duplex form;
having a Tm (melting temperature) of lower than 20° C.

having the nucleotides indicated here below in the sequence of the nucleotides, wherein “h” is A, C, T/U but not G; wherein “d” is A, G, T/U but not C, and wherein “w” is A or T/U, but not G or C:

| | | — | — | 1 | 2 | 3 | 4 | 5 | 6 | 7 | 8 | 9 | 10 | 11 | 12 | 13 | 14 | 15 | 16 | 17 | 18 | 19 | — | — | |
|---|---|---|---|---|---|---|---|---|---|---|---|---|---|---|---|---|---|---|---|---|---|---|---|---|---|
| mRNA | P′5 | A | A | | | A | | | | | | | U | | | h | | | | | | w | | | 3′-OH |
| si-ASense | OH-3′ | T | T | | | U | | | | | | | A | | | d | | | | | | w | | | 5′-P |
| si-Sense | P-5′ | | | | | A | | | | | | | U | | | h | | | | | | w | T | T | 3′-OH |

Production of agents intended herein, such as antisense agents and RNAi agents, can be carried out by any processes known in the art, such as inter alia partly or entirely by chemical synthesis (e.g., routinely known solid phase synthesis; an exemplary an non-limiting method for synthesising oligonucleotides on a modified solid support is described in U.S. Pat. No. 4,458,066; in another example, diethyl-phosphoramidites are used as starting materials and may be synthesised as described by Beaucage et al. 1981 (Tetrahedron Letters 22: 1859-1862)), or partly or entirely by biochemical (enzymatic) synthesis, e.g., by in vitro transcription from a nucleic acid construct (template) using a suitable polymerase such as a T7 or SP6 RNA polymerase, or by recombinant nucleic acid techniques, e.g., expression from a vector in a host cell or host organism. Nucleotide analogues can be introduced by in vitro chemical or biochemical synthesis. In an embodiment, the antisense agents of the invention are synthesised in vitro and do not include antisense compositions of biological origin, or genetic vector constructs designed to direct the in vivo synthesis of antisense molecules.

As noted elsewhere, an agent may comprise a recombinant nucleic acid comprising a sequence encoding one or more desired proteins, polypeptides or peptides operably linked to one or more regulatory sequences allowing for expression of said sequence or sequences encoding the proteins, polypeptides or peptides, e.g., in vitro, in a host cell, host organ and/or host organism (expression constructs). Such recombinant nucleic acid may be comprised in a suitable vector.

By “encoding” is meant that a nucleic acid sequence or part(s) thereof corresponds, by virtue of the genetic code of an organism in question to a particular amino acid sequence, e.g., the amino acid sequence of one or more desired proteins or polypeptides.

Preferably, a nucleic acid encoding one or more proteins, polypeptides or peptides may comprise one or more open reading frames (ORF) encoding said one or more proteins, polypeptides or peptides. An “open reading frame” or “ORF” refers to a succession of coding nucleotide triplets (codons) starting with a translation initiation codon and closing with a translation termination codon known per se, and not containing any internal in-frame translation termination codon, and potentially capable of encoding a protein, polypeptide or peptide. Hence, the term may be synonymous with “coding sequence” as used in the art.

An “operable linkage” is a linkage in which regulatory sequences and sequences sought to be expressed are connected in such a way as to permit said expression. For example, sequences, such as, e.g., a promoter and an ORF, may be said to be operably linked if the nature of the linkage between said sequences does not: (1) result in the introduction of a frame-shift mutation, (2) interfere with the ability of the promoter to direct the transcription of the ORF, (3) interfere with the ability of the ORF to be transcribed from the promoter sequence.

The precise nature of regulatory sequences or elements required for expression may vary between expression environments, but typically include a promoter and a transcription terminator, and optionally an enhancer.

Reference to a “promoter” or “enhancer” is to be taken in its broadest context and includes transcriptional regulatory sequences required for accurate transcription initiation and where applicable accurate spatial and/or temporal control of gene expression or its response to, e.g., internal or external (e.g., exogenous) stimuli. More particularly, “promoter” may depict a region on a nucleic acid molecule, preferably DNA molecule, to which an RNA polymerase binds and initiates transcription. A promoter is preferably, but not necessarily, positioned upstream, i.e., 5′, of the sequence the transcription of which it controls. Typically, in prokaryotes a promoter region may contain both the promoter per se and sequences which, when transcribed into RNA, will signal the initiation of protein synthesis (e.g., Shine-Dalgarno sequence).

In embodiments, promoters contemplated herein may be constitutive or inducible.

The terms “terminator” or “transcription terminator” refer generally to a sequence element at the end of a transcriptional unit which signals termination of transcription. For example, a terminator is usually positioned downstream of, i.e., 3′ of ORF(s) encoding a polypeptide of interest. For instance, where a recombinant nucleic acid contains two or more ORFs, e.g., successively ordered and forming together a multi-cistronic transcription unit, a transcription terminator may be advantageously positioned 3′ to the most downstream ORF.

The term “vector” generally refers to a nucleic acid molecule, typically DNA, to which nucleic acid segments may be inserted and cloned, i.e., propagated. Hence, a vector will typically contain one or more unique restriction sites, and may be capable of autonomous replication in a defined host or vehicle organism such that the cloned sequence is reproducible. Vectors may include, without limitation, plasmids, phagemids, bacteriophages, bacteriophage-derived vectors, PAC, BAC, linear nucleic acids, e.g., linear DNA, viral vectors, etc., as appropriate. Expression vectors are generally configured to allow for and/or effect the expression of nucleic acids or ORFs introduced thereto in a desired expression system, e.g., in vitro, in a host cell, host organ and/or host organism. For example, expression vectors may advantageously comprise suitable regulatory sequences.

As noted elsewhere, an agent may comprise an isolated or purified protein, polypeptide or peptide. Such may be suitably obtained through expression by host cells or host organisms, transformed with an expression construct encoding and configured for expression of said protein, polypeptide or peptide in said host cells or host organisms, followed by purification of the protein, polypeptide or peptide. Expression constructs are discussed above.

The terms “host cell” and “host organism” may suitably refer to cells or organisms encompassing both prokaryotes, such as bacteria, and eukaryotes, such as yeast, fungi, protozoan, plants and animals. Contemplated as host cells are inter alia unicellular organisms, such as bacteria (e.g., E. coli, Salmonella tymphimurium, Serratia marcescens, or Bacillus subtilis), yeast (e.g., Saccharomyces cerevisiae or Pichia pastoris), (cultured) plant cells (e.g., from Arabidopsis thaliana or Nicotiana tobaccum) and (cultured) animal cells (e.g., vertebrate animal cells, mammalian cells, primate cells, human cells or insect cells). Contemplated as host organisms are inter alia multi-cellular organisms, such as plants and animals, preferably animals, more preferably warm-blooded animals, even more preferably vertebrate animals, still more preferably mammals, yet more preferably primates; particularly contemplated are such animals and animal categories which are non-human.

The various active agents of the present disclosure or pharmaceutically acceptable derivatives thereof, may be formulated into pharmaceutical compositions or formulations with one or more pharmaceutically acceptable carriers/excipients.

The term “pharmaceutically acceptable” as used herein is consistent with the art and means compatible with the other ingredients of a pharmaceutical composition and not deleterious to the recipient thereof.

As used herein, “carrier” or “excipient” includes any and all solvents, diluents, buffers (such as, e.g., neutral buffered saline, phosphate buffered saline, or optionally Tris-HCl, acetate or phosphate buffers), solubilisers (such as, e.g., Tween 80, Polysorbate 80), colloids, dispersion media, vehicles, fillers, chelating agents (such as, e.g., EDTA or glutathione), amino acids (such as, e.g., glycine), proteins, disintegrants, binders, lubricants, wetting agents, emulsifiers, sweeteners, colorants, flavourings, aromatisers, thickeners, agents for achieving a depot effect, coatings, antifungal agents, preservatives (such as, e.g., Thimerosal, benzyl alcohol), antioxidants (such as, e.g., ascorbic acid, sodium metabisulfite), tonicity controlling agents, absorption delaying agents, adjuvants, bulking agents (such as, e.g., lactose, mannitol) and the like. The use of such media and agents for pharmaceutical active substances is well known in the art. Except insofar as any conventional media or agent is incompatible with the active substance, its use in the therapeutic compositions may be contemplated. Suitable pharmaceutical carriers are described inter alia in Remington's Pharmaceutical Sciences, 18th ed., Mack Publishing Co., Easton, Pa. (1990).

Illustrative, non-limiting carriers for use in formulating the pharmaceutical compositions include, for example, oil-in-water or water-in-oil emulsions, aqueous compositions with or without inclusion of organic co-solvents suitable for intravenous (IV) use, liposomes or surfactant-containing vesicles, particulate preparations with polymeric compounds such as inter alia polylactic acid or polyglycolic acid, microspheres, microbeads and microsomes, powders, tablets, capsules, suppositories, aqueous suspensions, aerosols, and other carriers apparent to one of ordinary skill in the art.

Pharmaceutical carriers may comprise sterile liquids, such as water and oils, including those of petroleum, animal, vegetable or synthetic origin, such as peanut oil, soybean oil, mineral oil, sesame oil and the like.

Pharmaceutical compositions of the invention may be formulated for essentially any route of administration, such as without limitation, oral administration (such as, e.g., oral ingestion or inhalation), intranasal administration (such as, e.g., intranasal inhalation or intranasal mucosal application), pulmonary (such as, e.g., by inhalation or insufflation of powders or aerosols), parenteral administration (such as, e.g., subcutaneous, intravenous, intra-arterial, intramuscular, intraperitoneal, or intrasternal injection or infusion, or intracranial, e.g., intrathecal or intraventricular administration), intra-osseous and/or intra-lesional administration, epidermal and transdermal, or transmucosal (such as, e.g., oral, sublingual, intranasal) administration, topical administration (including inter alia ophthalmic administration), rectal, vaginal or intra-tracheal instillation, and the like. In this way, the therapeutic effects attainable by the methods and compositions of the invention can be, for example, systemic, local, tissue-specific, etc., depending of the specific needs of a given application of the invention.

For example, for oral administration, pharmaceutical compositions may be formulated in the form of pills, tablets, lacquered tablets, coated (e.g., sugar-coated) tablets, granules, hard and soft gelatin capsules, aqueous, alcoholic or oily solutions, syrups, emulsions or suspensions. In an example, without limitation, preparation of oral dosage forms may be is suitably accomplished by uniformly and intimately blending together a suitable amount of the active compound in the form of a powder, optionally also including finely divided one or more solid carrier, and formulating the blend in a pill, tablet or a capsule. Exemplary but non-limiting solid carriers include calcium phosphate, magnesium stearate, talc, sugars (such as, e.g., glucose, mannose, lactose or sucrose), sugar alcohols (such as, e.g., mannitol), dextrin, starch, gelatin, cellulose, polyvinylpyrrolidine, low melting waxes and ion exchange resins. Compressed tablets containing the pharmaceutical composition can be prepared by uniformly and intimately mixing the active ingredient with a solid carrier such as described above to provide a mixture having the necessary compression properties, and then compacting the mixture in a suitable machine to the shape and size desired. Moulded tablets maybe made by moulding in a suitable machine, a mixture of powdered compound moistened with an inert liquid diluent. Suitable carriers for soft gelatin capsules and suppositories are, for example, fats, waxes, semisolid and liquid polyols, natural or hardened oils, etc.

For example, for oral or nasal aerosol or inhalation administration, pharmaceutical compositions may be formulated with illustrative carriers, such as, e.g., as in solution with saline, polyethylene glycol or glycols, DPPC, methylcellulose, or in mixture with powdered dispersing agents, further employing benzyl alcohol or other suitable preservatives, absorption promoters to enhance bioavailability, fluorocarbons, and/or other solubilising or dispersing agents known in the art. Suitable pharmaceutical formulations for administration in the form of aerosols or sprays are, for example, solutions, suspensions or emulsions of the compounds of the invention or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant. Illustratively, delivery may be by use of a single-use delivery device, a mist nebuliser, a breath-activated powder inhaler, an aerosol metered-dose inhaler (MDI) or any other of the numerous nebuliser delivery devices available in the art. Additionally, mist tents or direct administration through endotracheal tubes may also be used.

Examples of carriers for administration via mucosal surfaces depend upon the particular route, e.g., oral, sublingual, intranasal, etc. When administered orally, illustrative examples include pharmaceutical grades of mannitol, starch, lactose, magnesium stearate, sodium saccharide, cellulose, magnesium carbonate and the like, with mannitol being preferred. When administered intranasally, illustrative examples include polyethylene glycol, phospholipids, glycols and glycolipids, sucrose, and/or methylcellulose, powder suspensions with or without bulking agents such as lactose and preservatives such as benzalkonium chloride, EDTA. In a particularly illustrative embodiment, the phospholipid 1,2 dipalmitoyl-sn-glycero-3-phosphocholine (DPPC) is used as an isotonic aqueous carrier at about 0.01-0.2% for intranasal administration of the compound of the subject invention at a concentration of about 0.1 to 3.0 mg/ml.

For example, for parenteral administration, pharmaceutical compositions may be advantageously formulated as solutions, suspensions or emulsions with suitable solvents, diluents, solubilisers or emulsifiers, etc. Suitable solvents are, without limitation, water, physiological saline solution or alcohols, e.g. ethanol, propanol, glycerol, in addition also sugar solutions such as glucose, invert sugar, sucrose or mannitol solutions, or alternatively mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid. The compounds and pharmaceutically acceptable salts thereof of the invention can also be lyophilised and the lyophilisates obtained used, for example, for the production of injection or infusion preparations. For example, one illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

Where aqueous formulations are preferred, such may comprise one or more surfactants. For example, the composition can be in the form of a micellar dispersion comprising at least one suitable surfactant, e.g., a phospholipid surfactant. Illustrative examples of phospholipids include diacyl phosphatidyl glycerols, such as dimyristoyl phosphatidyl glycerol (DPMG), dipalmitoyl phosphatidyl glycerol (DPPG), and distearoyl phosphatidyl glycerol (DSPG), diacyl phosphatidyl cholines, such as dimyristoyl phosphatidylcholine (DPMC), dipalmitoyl phosphatidylcholine (DPPC), and distearoyl phosphatidylcholine (DSPC); diacyl phosphatidic acids, such as dimyristoyl phosphatidic acid (DPMA), dipahnitoyl phosphatidic acid (DPPA), and distearoyl phosphatidic acid (DSPA); and diacyl phosphatidyl ethanolamines such as dimyristoyl phosphatidyl ethanolamine (DPME), dipalmitoyl phosphatidyl ethanolamine (DPPE) and distearoyl phosphatidyl ethanolamine (DSPE). Typically, a surfactant:active substance molar ratio in an aqueous formulation will be from about 10:1 to about 1:10, more typically from about 5:1 to about 1:5, however any effective amount of surfactant may be used in an aqueous formulation to best suit the specific objectives of interest.

When rectally administered in the form of suppositories, these formulations may be prepared by mixing the compounds according to the invention with a suitable non-irritating excipient, such as cocoa butter, synthetic glyceride esters or polyethylene glycols, which are solid at ordinary temperatures, but liquidify and/or dissolve in the rectal cavity to release the drug.

Suitable carriers for microcapsules, implants or rods are, for example, copolymers of glycolic acid and lactic acid.

One skilled in this art will recognize that the above description is illustrative rather than exhaustive. Indeed, many additional formulations techniques and pharmaceutically-acceptable excipients and carrier solutions are well-known to those skilled in the art, as is the development of suitable dosing and treatment regimens for using the particular compositions described herein in a variety of treatment regimens.

The pharmaceutical compositions may comprise further components useful in the repair of bone wounds and defects. For example, such components may include without limitation bone morphogenetic proteins, bone matrix (e.g., bone matrix produced in vitro by cells or by other methods), hydroxyapatite/tricalcium phosphate particles (HA/TCP), cement (e.g., hydroxyapatite; mono-, bi-, or tri-calcium phosphate), gelatine, poly-lactic acid, poly-lactic glycolic acid, a glycosaminoglycan, hyaluronic acid, chitosan, a polysaccharide, poly-L-lysine, and collagen. For instance, such components may include without limitation bone morphogenetic proteins, bone matrix, HA/TCP, gelatine, poly-lactic acid, poly-lactic glycolic acid, hyaluronic acid, chitosan, poly-L-lysine, and collagen. The pharmaceutical composition can further include or be co-administered with a complementary bioactive factor such as a bone morphogenetic protein, such as BMP-2, BMP-7 or BMP-4, platelet-derived growth factor or any other growth factor. Other potential accompanying components include inorganic sources of calcium or phosphate suitable for assisting bone regeneration (WO 00/07639).

Further, there are several well-known methods of introducing nucleic acids (e.g., antisense and RNAi agents) into animal cells, any of which may be used herein. At the simplest, the nucleic acid can be directly injected into the target cell/target tissue. Other methods include fusion of the recipient cell with bacterial protoplasts containing the nucleic acid, the use of compositions like calcium chloride, rubidium chloride, lithium chloride, calcium phosphate, DEAE dextran, cationic lipids or liposomes or methods like receptor-mediated endocytosis, biolistic particle bombardment (“gene gun” method), infection with viral vectors, electroporation, and the like. Other techniques or methods which are suitable for delivering nucleic acid molecules to target cells include the continuous delivery of an NA molecule from poly (lactic-Co-Glycolic Acid) polymeric microspheres or the direct injection of protected (stabilized) NA molecule(s) into micropumps delivering the product. Another possibility is the use of implantable drug-releasing biodegradable micropsheres. Also envisaged is encapsulation of NA in various types of liposomes (immunoliposomes, PEGylated (immuno) liposomes), cationic lipids and polymers, nanoparticules or dendrimers, poly (lactic-Co-Glycolic Acid) polymeric microspheres, implantable drug-releasing biodegradable microspheres, etc; and co-injection of NA with protective agent like the nuclease inhibitor aurintricarboxylic acid. It shall be clear that also a combination of different above-mentioned delivery modes or methods may be used.

Further ways of delivery of nucleic acids such as antisense agents and RNAi agents may employ previously published methods. For example, intracellular delivery of the nucleic acids may be via a composition comprising an admixture of the nucleic acid molecule and an effective amount of a block copolymer. An example of this method is described in US 2004/0248833.

Other methods of delivery of nucleic acids to the nucleus are described in Mann et al. 2001 (Proc Natl Acad Science 98(1): 42-47) and in Gebski et al. 2003 (Human Molecular Genetics 12(15): 1801-1811).

A method for introducing a nucleic acid molecule into a cell by way of an expression vector either as naked DNA or complexed to lipid carriers, is described in U.S. Pat. No. 6,806,084.

It may be desirable to deliver a nucleic acid molecule in a colloidal dispersion system. Colloidal dispersion systems include macromolecule complexes, nanocapsules, microspheres, beads, and lipid-based systems including oil-in-water emulsions, micelles, mixed micelles, and liposomes or liposome formulations. Liposomes are artificial membrane vesicles which are useful as delivery vehicles in vitro and in vivo. These formulations may have net cationic, anionic or neutral charge characteristics and are useful characteristics with in vitro, in vivo and ex vivo delivery methods. It has been shown that large unilamellar vesicles (LUV), which range in size from 0.2-4.0 PHI.m can encapsulate a substantial percentage of an aqueous buffer containing large macromolecules. RNA, and DNA can be encapsulated within the aqueous interior and be delivered to cells in a biologically active form (Fraley et al. 1981 (Trends Biochem ScL 6: 77).

In order for a liposome to be an efficient gene transfer vehicle, the following characteristics should be present: (1) encapsulation of the nucleic acid molecule of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino et al. 1988 (Biotechniques 6: 682).

The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations.

Alternatively, the nucleic acid molecule may be combined with other pharmaceutically acceptable carriers or diluents to produce a pharmaceutical composition. Suitable carriers and diluents include isotonic saline solutions, for example phosphate-buffered saline. The composition may be formulated for parenteral, intramuscular, intravenous, subcutaneous, intraocular, oral or transdermal administration.

The routes of administration described are intended only as a guide since a skilled practitioner will be able to determine readily the optimum route of administration and any dosage for any particular animal and condition. Multiple approaches for introducing functional new genetic material into cells, both in vitro and in vivo have been attempted (Friedmann 1989 (Science 244: 1275-1280)). These approaches include integration of the gene to be expressed into modified retroviruses (Friedmann 1989, supra; Rosenberg. Cancer Res. 1991, vol. 51(18), 5074S-79S); integration into non-retrovirus vectors (Rosenfeld et al. Cell, 1992, vol. 68, 143-55; Rosenfeld et al. Science,1991, vol. 252, 431-4); or delivery of a transgene linked to a heterologous promoter-enhancer element via liposomes (Friedmann 1989, supra; Brigham et al. Am. J. Med. Sci., 1989, vol. 298, 278-81; Nabel et al. Science, 1990, vol. 249, 1285-8; Hazinski et al. Am. J. Resp. Cell. Molec. Biol., 1991, vol. 4, 206-9; Wang & Huang. Proc. Natl. Acad. Sci. USA., 1987, vol. 84, 7851-5); coupled to ligand-specific, cation-based transport systems (Wu & Wu. J. Biol. Chem., 1988, vol. 263, 14621-4)) or the use of naked DNA, expression vectors (Nabel et al. 1990, supra; Wolff et al. Science, 1990, vol. 247, 1465-8). Direct injection of transgenes into tissue produces only localized expression (Rosenfeld. 1992, supra; Rosenfeld et al. 1991, supra; Brigham et al. 1989, supra; Nabel 1990, supra; Hazinski et al. 1991, supra). The Brigham et al. group (Am. J. Med. Sci., 1989, vol. 298, 278-81; Clin. Res., 1991, vol. 39, abstract) have reported in vivo transfection only of lungs of mice following either intravenous or intratracheal administration of a DNA liposome complex. An example of a review article of human gene therapy procedures is: Anderson. Science, 1992, vol. 256, 808-13.

The pharmaceutical formulations as disclosed herein, which may conveniently be presented in unit dosage form, may be prepared according to conventional techniques well known in the pharmaceutical industry. Such techniques may generally include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The present active agents may be used alone or in combination with any therapies known in the art for treatment of impaired bone healing, such as, e.g., BMP-2 (“combination therapy”). Combination therapies as contemplated herein may comprise the administration of at least one active agent of the present invention and at least one other pharmaceutically or biologically active ingredient. Said present active agent(s) and said pharmaceutically or biologically active ingredient(s) may be administered in either the same or different pharmaceutical formulation(s), simultaneously or sequentially in any order.

In further examples, agents as described herein may be combined with, or compositions as described herein may further comprise, cells having therapeutic effect on impaired bone fracture healing. By means of example, such cells may be mesenchymal stem cells (MSC), bone marrow stromal cells (BMSC), osteoblastic cells such as pre-osteoblasts or osteoblasts, or osteocytes. Advantageously, such cells may be autologous, or more preferably may be allogeneic.

The dosage or amount of the present active agents used, optionally in combination with one or more other active compound to be administered, depends on the individual case and is, as is customary, to be adapted to the individual circumstances to achieve an optimum effect. Thus, it depends on the nature and the severity of the disorder to be treated, and also on the sex, age, body weight, general health, diet, mode and time of administration, and individual responsiveness of the human or animal to be treated, on the route of administration, efficacy, metabolic stability and duration of action of the compounds used, on whether the therapy is acute or chronic or prophylactic, or on whether other active compounds are administered in addition to the agent(s) of the invention.

Without limitation, depending on the type and severity of the disease, a typical daily dosage might range from about 1 ng/kg to 100 mg/kg of body weight or more, depending on the factors mentioned above. For repeated administrations over several days or longer, depending on the condition, the treatment is sustained until a desired suppression of disease symptoms occurs. A preferred dosage of the active substance of the invention may be in the range from about 0.05 mg/kg to about 10 mg/kg of body weight. Thus, one or more doses of about 0.5 mg/kg, 2.0 mg/kg, 4.0 mg/kg or 10 mg/kg (or any combination thereof) may be administered to the patient. Such doses may be administered intermittently, e.g., every week or every two or three weeks.

In an embodiment, a pharmaceutical composition may comprise between about 10 nM and about 1 μM, preferably between about 20 nM and about 600 nM, such as, e.g., about 100 nM or about 200 nM, or about 300 nM, or about 400 nM or about 500 nM of antisense agent or RNAi agent as taught herein.

In a non-limiting embodiment, for administration at the site of impaired fracture healing between about 1 ng and about 500 μg of SDF-1 protein may be administered.

In a non-limiting embodiment, for administration at the site of impaired fracture healing between about 1 ng and about 1 mg of IL-8 protein may be administered.

In a non-limiting embodiment, for administration at the site of impaired fracture healing between about 1 ng and about 100 μg of IL-6 protein may be administered.

It is apparent that there have been provided in accordance with the invention products, methods and uses that provide for substantial advantages as set forth above. While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and broad scope of the appended claims.

The above aspects and embodiments are further supported by the following non-limiting examples.

### EXAMPLES

### Example 1

### Measurement of Altered Protein Levels in Serum/Plasma

Two groups of subjects entered the study: (1) healthy volunteers (HV), (2) patients with impaired fracture healing, in particular with non-union fractures (NU). The patient population was distributed as follows:

| | Healthy | | |
|---|---|---|---|
| | volunteer (HV) | Non-union (NU) | P |
| Number of subjects | 79 | 20 | |
| Mean age (years ± SD) | 32 ± 10 | 43 ± 16 | 0.012 |
| Sex (%) Female | 67 | 20 | |
| Fracture site | — | Long bones | |
| Delay (months ± SD) | — | 25 ± 15 | |

The mean age in the two groups varied between thirty and forty years old. Non-union patients were older (P=0.012) and were mostly male. However, the results stayed unchanged independent of gender and age. The bone sites were in long bones (radius, humerus, fibula, tibia and cubitus) except 2 fractures of the metatarsus and 2 fractures of the calcaneum. The delay between the fracture and sample harvesting varied around 25 months with a standard deviation of 15 months.

To identify proteins having altered presence in non-unions, sera were collected in dry tubes and plasma were collected in heparin or EDTA tubes, centrifuged, aliquoted and frozen at −20° C. until use. These were used to determine the level of growth factors and proteins using enzyme-linked immunosorbent assays (ELISA).

Stromal-derived factor one was measured in the plasma (SDF-1/CXCL12, Duoset, R&D Systems, Abingdon, United Kingdom). The following biomarkers were measured in the serum: platelet-derived growth factor-BB (PDGF-BB, Quantikine™, R&D Systems, Abingdon, United Kingdom), interleukin-8 (IL8/CXCL8, Quantikine™, R&D Systems, Abingdon, United Kingdom) and interleukin 6 (IL-6, Quantikine™, R&D Systems, Abingdon, United Kingdom).

All continuous values are expressed as medians±standard error of the mean (SEM), all reported P values are 1-sided, and statistical significance is assessed at the 10% level. The normality of distribution was tested with a Kolmogorov-Smirnov test. When the Kolmogorov-Smirnov test failed, differences between groups were analyzed by a Mann-Whitney test.

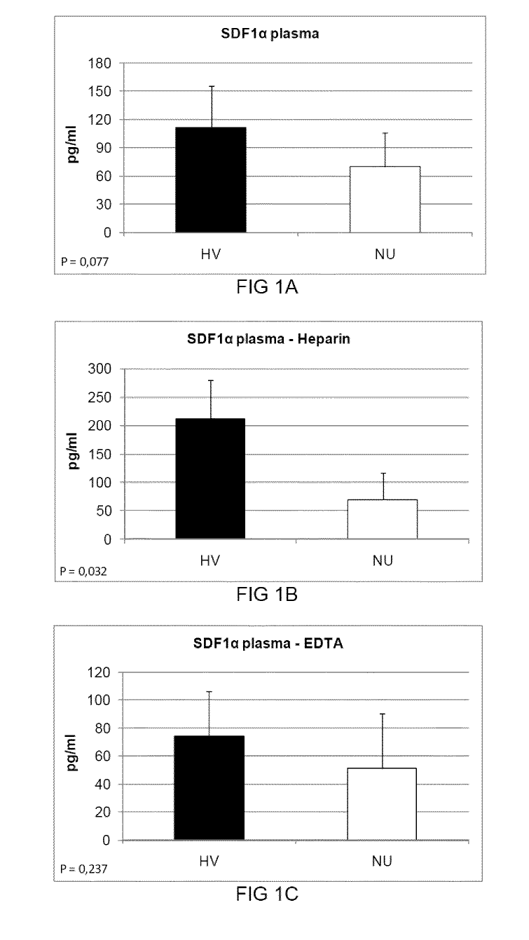

When compared to HV, in NU patients, the plasma level of SDF-1 was decreased (FIG. 1A). The decrease was more pronounced in plasma collected in heparin tubes compared with plasma collected in EDTA tubes (FIGS. 1B and 1C).

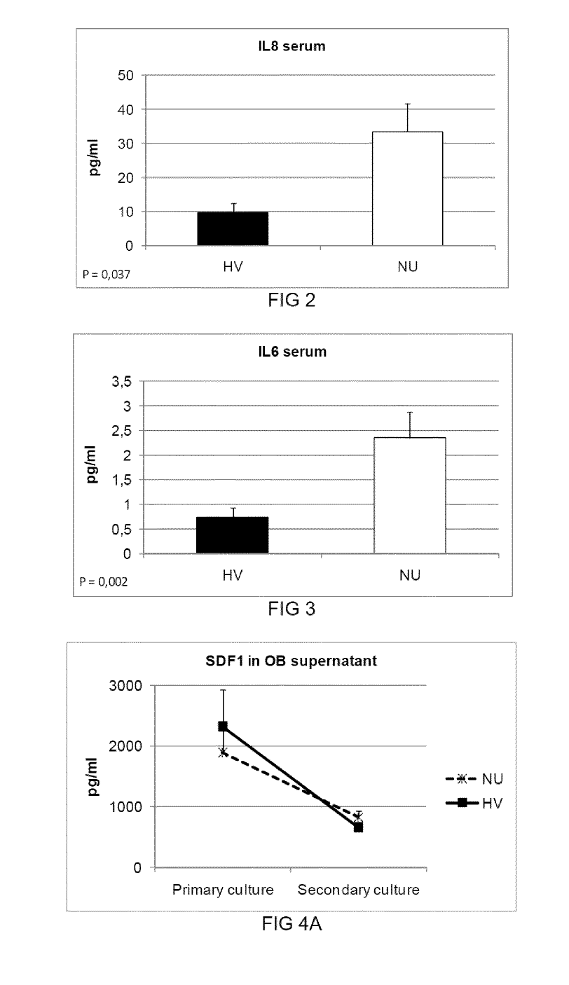

When compared with healthy volunteers (HV), the serum level of IL-8 was increased in NU patients (FIG. 2).

When compared with healthy volunteers (HV), the serum level of IL-6 tended to be increased in NU patients (FIG. 3).

### Example 2

### Culturing Cells from Subjects

The presence of these proteins showed alterations also when measured in cells or in the supernatant of cells obtained from subjects and cultured in vitro, preferably from osteoblastic cells (OB) or mesenchymal stem cells (MSC). The following provides suitable protocols for isolation, differentiation and culture of such cells.

Twenty to sixty ml of heparinised bone marrow (BM) was obtained from iliac crest distant from the fracture site. BM was mixed with phosphate-buffered saline (PBS:BM ratio (v:v): 2) and layered on density gradient Ficoll solution. After centrifugation, mononuclear cells were harvested from the interface and washed twice in PBS. The cells were plated at 1.43×10⁶ cells/25 cm² flasks in two different media; (1) a mesenchymal medium composed of DMEM, 10% foetal bovine serum, 1% L-glutamine, 1% penicillin and 1% streptomycin; (2) an osteogenic medium. Cells were maintained in a 37° C. humidified atmosphere containing 5% CO₂. Medium changes were done every 2 to 3 days. When confluent, cells of the primary culture were detached and replated for the secondary culture. The supernatants of these 2 culture passages were collected and frozen until use.

The ELISA reagents protocols used for blood samples are applied to the cell supernatants with routine adaptation.

### Example 3

### Autocrine/Paracrine Activity of Osteoblastic Cells and Mesenchymal Stem Cells

To study the autocrine/paracrine activity of osteoprogenitor cells in impaired bone fracture healing, the level of growth factors secreted in supernatant osteoblastic cell (OB) or mesenchymal cell (MSC) culture was assessed by ELISA. The following growth factors were measured; stromal-derived factor one (SDF-1/CXCL12, Duoset, R&D Systems, Abingdon, United Kingdom), and interleukin-6 (IL-6, Duoset, R&D Systems, Abingdon, United Kingdom). Values were expressed in pg/ml of supernatant.

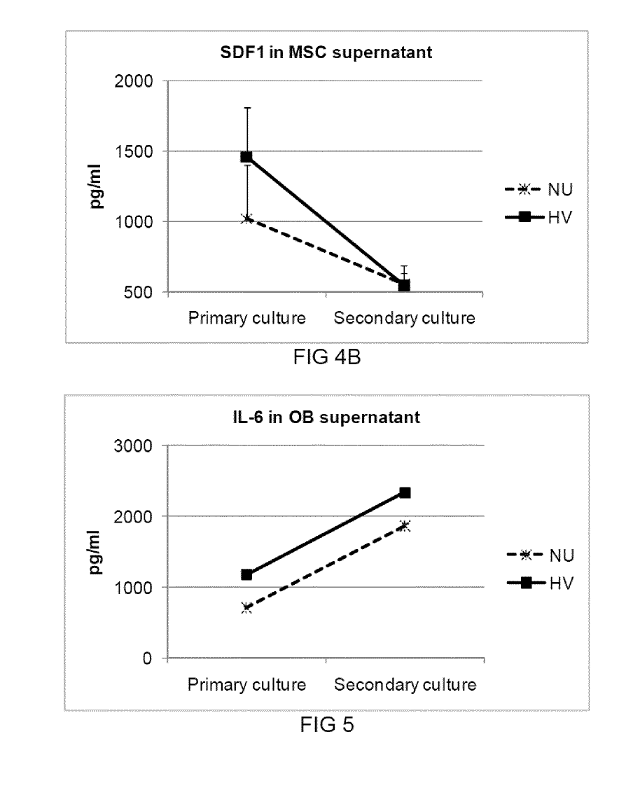

When compared with healthy volunteers (HV), SDF-1 was less secreted in supernatant of OB and MSC culture of non-union patients (NU) at the end of primary cell culture (FIGS. 4A and 4B).

Furthermore, IL-6 was less secreted in supernatant of OB culture of NU patients at the end of primary and secondary cell cultures when compared with HV (FIG. 5).

### Example 4

### Effect of Growth Factors SDF-1, IL-8 and/or IL-6 in Calvarial Defect Repair in Mice

The present example concerns the efficacy of SDF-1α, IL-8 and IL-6 each alone or in combination of any two or all three in administration in situ on bone repair in a murine model of calvarial defect.

7 groups of male adult mice (+/−25 g each) are included in the study (n=35). At day 0, after general anaesthesia, mice undergo a calvarial osteotomy: a large bone defect (2 mm of diameter) is performed.

Mice are randomly allocated into 7 groups to receive one of the following items:

Group 1: Vehicle solution composed of gelatin (50 μl, n=5)
Group 2: BMP2 (50 μg) in the vehicle solution (50 μl, n=5)
Group 3: SDF-1α (10 μg) in the vehicle solution (50 μl, n=5)
Group 4: IL-8 (1 μg) in the vehicle solution (50 μl, n=5)
Group 5: IL-6 (40 ng)+receptor of IL-6 (100 ng) in the vehicle solution (50 μl, n=5)
Group 6: SDF-1α (10 μg)+IL-8 (1 μg) in the vehicle solution (50 μl, n=5)
Group 7: SDF-1α (10 μg)+IL-6 (40 ng)+receptor of IL-6 (100 ng) in the vehicle solution (50 μl, n=5)
Group 8: SDF-1α (10 μg)+IL-8 (1 μg)+IL-6 (40 ng)+receptor of IL-6 (100 ng) in the vehicle solution (50 μl, n=5)

Growth factors are administered into the calvarial defect, in sterile condition, just after the osteotomy surgery (before suturing the lesion). After 2, 4 and 6 weeks, bone formation is assessed by CT-scan imaging. The bone repair progress is determined as the presence of mineralized tissue in the osteotomy site. At the end of the protocol, mice are euthanized and samples are taken for immunohistochemistry analysis.

BMP-2 is used in the experiment as positive control of bone repair. It has indeed been shown that, in the absence of any matrix, BMP-2 was able to induce formation of bone at a concentration of 100 to 200 μg after subcutaneous delivery (Wang et al. PNAS, 1990, vol. 87, 2220-2224). The same observations have been made when 80 μg rhBMP2 were injected into bone fracture (Einhorn et al. J. Bone Joint Surg Am., 2003, vol. 85-A, 1425-1435).

It is awaited that the mice to which SDF-1α, IL-8 and/or IL-6 are administered display significantly improved bone formation and healing of the calvarial defect compared to respective control mice without administration of SDF-1α, IL-8 and/or IL-6.

### Example 5

### Tibial Intramedullary Administration of SDF-1α and/or Osteoblastic Stem Cells in Nude Mice

The present example concerns the efficacy of bone formation following injection of SDF-1α with or without osteoblastic cells in the tibial intramedullary cavity.

Six groups of male adult nude mice (±25 g each) are included in the study (n=30). At day 0, after general anaesthesia, mice are randomly allocated to receive intratibial administration of one of the following items:

Group 1: Vehicle solution containing PBS+5% HAS (40 μl, n=5)
Group 2: BMP2 (50 μg) in the vehicle solution (40 μl, n=5)
Group 3: SDF-1α (10 μg) in the vehicle solution (40 μl, n=5)
Group 4: IL-8 (1 μg) in the vehicle solution (40 μl, n=5)
Group 5: IL-6 (40 ng)+receptor of IL-6 (100 ng) in the vehicle solution (40 μl, n=5)
Group 6: Osteoblastic stem cells (1×10⁶ cells) in the vehicle solution (40 μl, n=5)
Group 7: SDF-1α (10 μg)+osteoblastic stem cells (1×10⁶ cells) in the vehicle solution (40 μl, n=5)
Group 8: IL-8 (1 μg)+osteoblastic stem cells (1×10⁶ cells) in the vehicle solution (40 μl, n=5)
Group 9: IL-6 (40 ng)+receptor of IL-6 (100 ng)+osteoblastic stem cells (1×10⁶ cells) in the vehicle solution (40 μl, n=5)
Group 10: SDF-1α (10 μg)+11-8 (1 μg)+IL-6 (40 ng)+receptor of IL-6 (100 ng)+osteoblastic stem cells (1×10⁶ cells) in the vehicle solution (40 μl, n=5)

The administrations are performed in sterile condition under a laminar airflow. After 4 and 8 weeks, bone formation is assessed by CT-scan imaging. The presence of bone formation is determined as the presence of mineralized tissue into the intramedullary cavity. At the end of the protocol, mice are euthanized and samples are taken for immuno-histochemistry analysis.

It is awaited that the mice to which SDF-1α is administered display significantly improved bone formation compared to respective control mice without administration of SDF-1α.

### Example 6

### Efficacy of SDF-1α and/or Osteoblastic Stem Cells Administration in a Murine Model of Osteotomy

The present example concerns the efficacy of the administration of SDF-1α with or without osteoblastic cells on bone repair in a murine model of osteotomy.

Six groups of male adult nude rats (+/−250 g each) are included in the study (n=18). At day 0, after general anaesthesia, rats undergo tibial osteotomy: a large bone defect (5 mm) is performed and the edges of the bony fragments are cauterized.

At day 1 the rats are randomly allocated into six groups to receive, at the site of osteotomy, one of the following items:

Group 1: Vehicle solution containing PBS+5% HAS (40 μl, n=5)
Group 2: BMP2 (50 μg) in the vehicle solution (40 μl, n=5)
Group 3: SDF-1α low dose (1 ng) in the vehicle solution (40 μl, n=5)
Group 4: IL-8 (1 μg) in the vehicle solution (40 μl, n=5)
Group 5: IL-6 (40 ng)+receptor of IL-6 (100 ng) in the vehicle solution (40 μl, n=5)
Group 6: Osteoblastic stem cells (1×10⁶ cells) in the vehicle solution (40 μl, n=5)
Group 7: SDF-1α (1 μg)+osteoblastic stem cells (1×10⁶ cells) in the vehicle solution (40 μl, n=5)
Group 8: IL-8 (1 μg)+osteoblastic stem cells (1×10⁶ cells) in the vehicle solution (40 μl, n=5)
Group 9: IL-6 (40 ng)+receptor of IL-6 (100 ng)+osteoblastic stem cells (1×10⁶ cells) in the vehicle solution (40 μl, n=5)
Group 10: SDF-1α (1 μg)+IL-8 (1 μg)+IL-6 (40 ng)+receptor of IL-6 (100 ng)+osteoblastic stem cells (1×10⁶ cells) in the vehicle solution (40 μl, n=5)

The administrations are performed in sterile condition under a laminar airflow. After 2, 4 and 6 weeks, bone formation is assessed by CT-scan imaging. The bone repair progress is determined as the presence of mineralized tissue in the osteotomy site. At the end of the protocol, rats are euthanized and samples are taken for immunohistochemistry analysis.

It is awaited that the rats to which SDF-1α is administered display significantly enhanced bone formation and healing of the tibial osteotomy defect compared to respective control rats without administration of SDF-1α, IL-8 and/or IL-6.

### Example 7

### Bone Formation In Vivo in a Mice Model for Non-Union Fractures

Bone formation was studied using a murine model of calvarial defect. A composition comprising an IL-8 peptide (30 μg) was administered by injection/deposition into the calvarial defect. The composition further comprised a gel-forming material such as porcine collagen. The IL-8 peptides were human IL-8 peptide having SEQ ID No. 1 (Table 1) and human IL-8 peptide having SEQ ID No. 2 (Table 1); these IL-8 peptides are also referred to herein as “long IL-8 peptide” and “short IL-8” peptide respectively. The long IL-8 peptide comprised 77 amino acids namely amino acids 3 to 79. The short IL-8 peptide comprised 72 amino acids namely amino acids 8 to 79.

Amino acid sequences of IL-8 peptides

| | | SEQ ID |
|---|---|---|
| IL-8 peptide | Sequence | No. |
| Long IL-8 | AVLPRSAKEL RCQCIKTYSK PFHPKFIKEL RVIESGPHCA NTEIIVKLSD | 1 |
| peptide | GRELCLDPKE NWVQRVVEKF LKRAENS | |
| Short IL-8 | SAKELRCQCI KTYSKPFHPK FIKELRVIES GPHCANTEII VKLSDGRELC | 2 |
| peptide | LDPKENWVQR VVEKFLKRAE NS | |

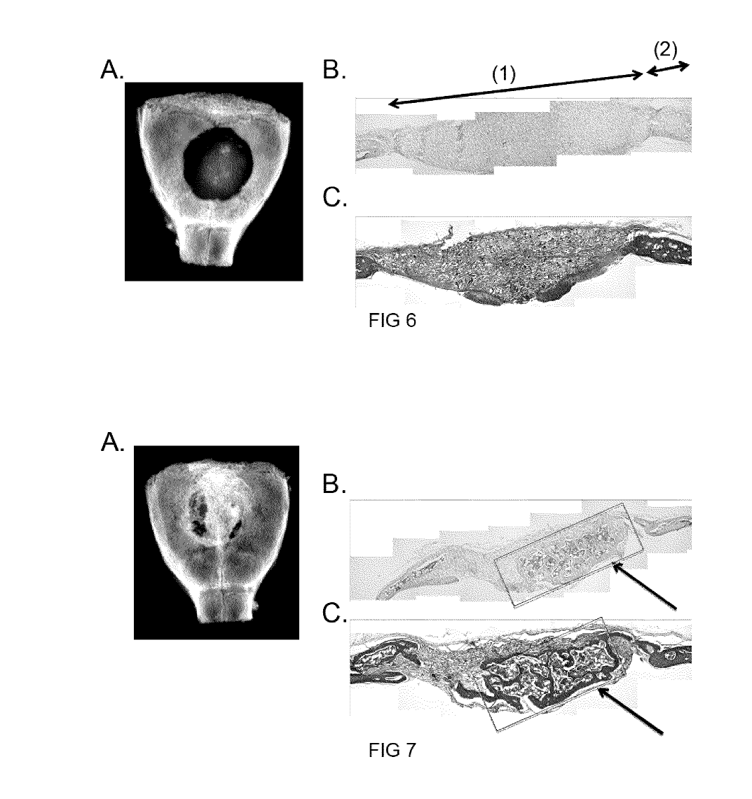

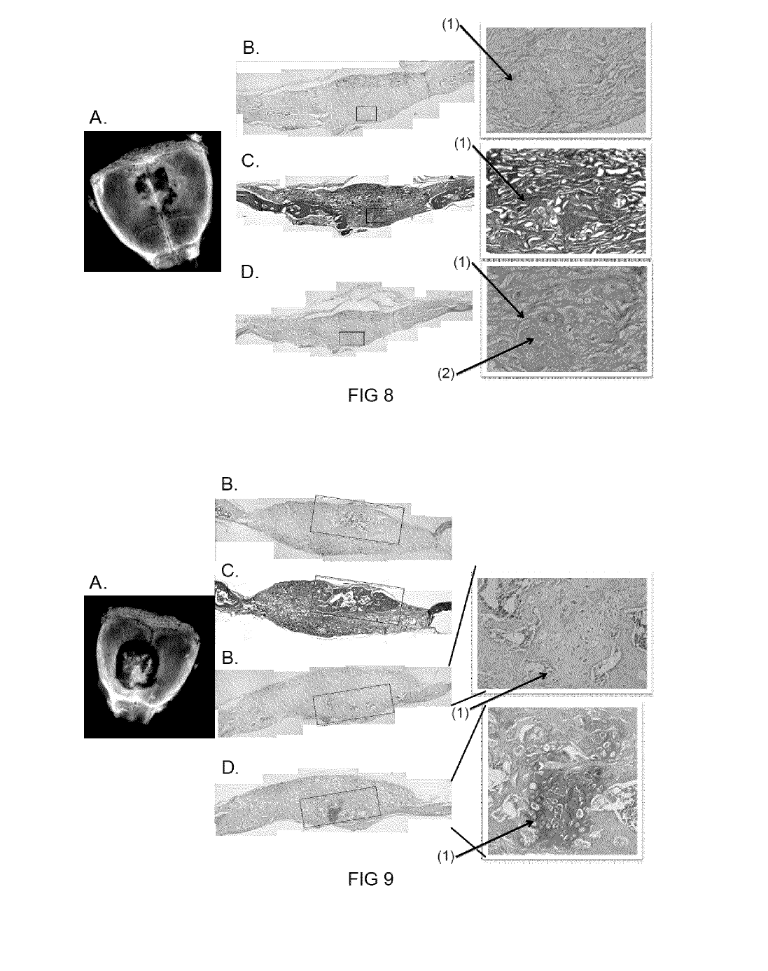

After trepanation, four mice received the composition comprising the long IL-8 peptide and three mice received the composition comprising the short IL-8 peptide. Vehicle (PBS-HSA) and BMP-2 (2, 5, 10, and 20 mg) were used as negative and positive control respectively. Results for negative control and positive control are shown in FIG. 6 and FIG. 7 respectively. Results for the composition comprising the long IL-8 peptide and the composition comprising the short IL-8 peptide are illustrated in FIG. 8 and FIG. 9 respectively. Mice were sacrificed 4 or 6 weeks after trepanation. Bone formation was assessed by CT-scan imaging. The bone repair progress was determined as the presence of mineralized tissue in the osteotomy site. At the end of the protocol, mice were euthanized and samples were taken for immunochemistry analysis. Masson's trichrome was used to visualize collagen fibers; muscle colored red, and bone colored blue. Safranin-O was used to highlight hypertrophic chondrocytes.

The four mice that received the composition comprising the long IL-8 peptide and the three mice that received the composition comprising the short IL-8 peptide presented bone repair compared with vehicle (FIG. 6, FIG. 8 and FIG. 9). On the Hematoxylin-eosin and Masson's trichrome histological slides, new bone formation appeared denser for mice that received a composition comprising an IL-8 peptide compared with negative control (FIG. 6, FIG. 8 and FIG. 9). In FIGS. 6 to 9, boxes and arrows indicate the zones where the bone formation was observed. Note that for the treatment with the composition comprising the short IL-8 peptide (FIG. 9), two different levels in the calvarial defect are illustrated due to the fact that Masson's trichrome and Safranin-O staining were not performed on the same level.

On the histological slides (Hematoxylin-eosin, Masson's trichrome, Safranin-O), it was observed that both compositions comprising an IL-8 peptide induced osseous reparation of calvarial defect with the development of osteoid in the gel-forming material. In literature, it is accepted that the reparation of calvaria occurs by intra-membranous ossification. Unexpectedly, hypertrophic chondrocytes were also observed on histological slides revealing an endochondral ossification when using a composition comprising an IL-8 peptide (FIGS. 8 and 9). Therefore, these data provide in vivo evidence that each of the IL-8 peptides advantageously had a positive effect on bone repair in calvarial defects in mice. Bone calvarial defects in mice are a model for non-union fractures and hence, the presented data provide in vivo evidence that each of the IL-8 peptides advantageously had a positive effect on bone repair in non-union fractures. Due to the differences between normal bone fracture healing and non-union fractures characterised by a failure of fracture repair, the bone repair in the presence of a composition comprising an IL-8 peptide was an unexpected observation.

Conclusively, a more intense ossification was observed in the non-union fractures using a composition comprising an IL-8 peptide compared with negative control.

1. A method of treating impaired bone fracture healing, the method comprising a step of administering one or more pharmaceutical active ingredients selected from the group consisting of interleukin-8 (IL-8), a functional fragment of IL-8, a functional variant of IL-8, and an agonist of IL-8 receptor.

2. The method according to claim 1, wherein the impaired bone fracture healing is selected from the group consisting of non-union fracture, mal-union fracture, and delayed union fracture.

3. The method according to claim 1, wherein the pharmaceutical active ingredient is an IL-8 peptide or a functional variant thereof, wherein the IL-8 peptide comprises an amino acid sequence selected from SEQ ID No. 1 or SEQ ID No. 2.

4. The method according to claim 1, wherein the composition further comprises a gel-forming material.

5. The method according to claim 4, wherein the gel-forming material is collagen, a glyceride, a glycosaminoglycan, a polysaccharide, gelatine, poly-lactic acid, or poly-lactic glycolic acid.

6. The composition for use method according to claim 4, wherein the gel-forming material is collagen and the pharmaceutical active ingredient is an IL-8 peptide comprising an amino acid sequence selected from SEQ ID No. 1 or SEQ ID No. 2.

7. The method according to claim 1, wherein the composition is administered percutaneously, preferably by percutaneous injection.
